SEQUENCE LISTING (10 sequences)

SEQ ID NO: 1 - protein, 11 aa, Artificial
  Protein translocation domain
YGRKKRRQRRR

SEQ ID NO: 2 - protein, 9 aa, Artificial
  Protein translocation domain
RKKRRQRRR

SEQ ID NO: 3 - protein, 11 aa, Artificial
  Protein translocation domain
YARKARRQARR

SEQ ID NO: 4 - protein, 11 aa, Artificial
  Protein translocation domain
YARAAARQARA

SEQ ID NO: 5 - protein, 11 aa, Artificial
  Protein translocation domain
YARAARRAARR

SEQ ID NO: 6 - protein, 11 aa, Artificial
  Protein translocation domain
RARAARRAARA

SEQ ID NO: 7 - protein, 16 aa, Artificial
  Protein transduction domain
RQIKIWFQNRRMKWKK

SEQ ID NO: 8 - protein, 34 aa, Artificial
  Protein translocation domain
DAATATRGRSAASRPTERPRAPARSASRPRRPVE

SEQ ID NO: 9 - protein, 7 aa, Artificial
  Protein translocation domain
RRRRRRR

SEQ ID NO: 10 - protein, 33 aa, Artificial
  Protein translocation domain
YARAAARQARAYARAAARQARAYARAAARQARA

(19) United States
(12) Patent Application Publication
(10) Pub. No.: US 2012/0184035 A1
(43) Pub. Date: Jul. 19, 2012

(54) Methods and Compositions For Reprogramming Cells

(76) Inventors: Sadhana Agarwal, Cambridge, MA (US); Katerine L. Holton, Auburn, MA (US); Robert Lanza, Clinton, MA (US)

(21) Appl. No.: 13/006,064

(22) Filed: Jan. 13, 2011

(51) Int. Cl. C12N 5/07

(52) U.S. Cl. 435/375

(57) ABSTRACT

Methods and compositions are provided for reprogramming cells. In an exemplary embodiment, fibroblasts are reprogrammed to adopt a skeletal, cardiac, or smooth muscle cell fate. Cell and tissue therapies using said methods and compositions are also disclosed.

### BACKGROUND

1. Field of the Art

The present disclosure relates to methods and materials for reprogramming. Human somatic cells or human somatic cell nuclei may be reprogrammed using the disclosed materials and methods. These methods have application especially in the context of cell-based therapies, tissue transplantation, establishment of cell lines, and the production of genetically modified cells and chimeric or transgenic animals. In a preferred embodiment, reprogrammed fibroblasts adopt a muscle cell fate.

2. Description of Related Art

The transdifferentiation potential of adult cells has been receiving increasing attention (Eguchi and Kodama, 1993). Transdifferentiation is a physiological process that occurs during development but has also been described in a number of adult organs including liver, thyroid, mammary gland (Hay and Zuk, 1999), and kidney (Strutz et al., 1995; Ng et al., 1999). It has been shown that alteration of cell morphology and function can be induced artificially in vitro by treatment of cell cultures with cytoskeletal disruptors, hormones, and Calcium-ionophores. Alteration of cell fate can be induced artificially in vitro and there is a vast amount of published data describing trans-differentiation. For example, embryonic blastomeres can be induced to differentiate in the presence of micro filament inhibitors (Okado and Takahashi, 1988, 1990; Wu et al., 1990; Pratt et al., 1981). Supplementing growth media for somatic cells with cytoskeletal inhibitors (Brown and Benya, 1988; Takigawa et al., 1984; Shea, 1990; Tamai et al., 1999; Cohen et al., 1999; Fernandez-Valle et al., 1997; Yujiri et al., 1999; Ulloa and Avila, 1996; Ferreira et al., 1993; Sato et al., 1991; Zanetti and Solursh, 1984; Kishkina et al., 1983; Hamano and Asofsky, 1984; Holtzer et al., 1975; Cohen et al., 1999), Ca-ionophores (Shea, 1990; Sato et al., 1991), corticosteroids (Yeomans et al., 1976), and DMSO (Hallows and Frank, 1992), causes changes in cell shape and function. Mammary epithelial cells can be induced to acquire muscle-like shape and function (Paterson and Rudland, 1985), spleen cells can be induced to produce both IgM and IgG immunoglobulins (van der Loo et al., 1979), pancreatic exocrine duct cells can acquire insulin-secreting, endocrine, phenotype (Bouwens, 1998a, b), 3T3 cells into adipose cells (Pairault and Lasnier, 1987), mesenchymal cells into chondroblasts (Rosen et al., 1986), bone marrow cells into liver cells (Theise et al., 2000), islets into ductal cells (Yuan et al., 1996), muscle into 7 non-muscle cell types, including digestive, secretory, gland, nerve cells (Schmid and Alder, 1984), muscle into cartilage (Nathanson, 1986), neural cells into muscle (Wright, 1984), bone marrow into neuronal cells (Black, 2000).

### SUMMARY

In one aspect, the present disclosure provides methods of converting a somatic cell or somatic cell nucleus to a cell or nucleus of target type, wherein said target type may be skeletal muscle, cardiac muscle, or smooth muscle. Exemplary methods comprise: (a) culturing the somatic cell or somatic cell nucleus in the presence of a reprogramming composition, thereby converting the somatic cell or somatic cell nucleus into a cell or nucleus of the target type, wherein said reprogramming composition may comprise one or more reprogramming agents; and (b) identifying a cell or cell nucleus of the target type that results from the culture. The target type may be skeletal muscle.

The reprogramming composition may comprise an extract obtained from a muscle cell, an extract obtained from a myoblast cell, a muscle cell conditioned medium, or a myoblast cell conditioned medium. The extract or conditioned medium may be obtained from cells of the same type as the target type or from a different type of cells (e.g., another type of muscle cell or a progenitor of another type of muscle cell). In embodiments utilizing an extract, the extract may be a whole cell extract, nuclear extract, or cytoplasmic extract. The reprogramming composition may further comprise one or more exogenously added reprogramming agents. Optionally, the cell from which said extract may be obtained may be modified to produce or express elevated levels of one or more reprogramming agents.

The exogenously added reprogramming agents may comprise one or more polypeptides selected from the group consisting of: MyoD, IGF-1, aFGF, and Activin A, or may comprise one or more polypeptides selected from the group consisting of Activin A, aFGF, Akt1, Akt2, Bmp4, Capsulin, c-MET, one or more E-proteins, E12, E47, Eya2, FoxO3, Gli, HEB, Hepatocyte Growth Factor (HGF) or Scatter factor (SF), an antagonist of Id, IGF-1, Lbx1, an antagonist of Mdfi, MEF2, Mef2c, Mef2d, MEF3, Meis, Meox2, microRNA-1, microRNA-1-1, microRNA-1-2, microRNA-27, microRNA-133, microRNA-133a-1, microRNA-133a-2, microRNA-133b, microRNA-206, microRNA-208, microRNA-208b, microRNA-499, MRF4, My5, Myf5, Myh3, Myh6, Myh7, Myh7b, MyoD, Myogenin, MyoR, p38, Paraxis, Pax3, Pax7, Pbx, Pitx2, an antagonist of Pur-beta, Six1, Six4, Sonic Hedgehog, an antagonist of Hedgehog signal transduction or the Gli transcription factor, an antagonist of Sox6, an antagonist of Sp3, Sp1, TAF3, Tbx1, TRF3, Wnt, and Wnt ligand.

The method may further comprise prior to, concurrently with, and/or subsequently to step (a), culturing said somatic cell or somatic cell nucleus in the presence of one or more cytoskeletal disruptors, chromatin remodeling agents, and/or transcription modifiers.

The method may further comprise prior to, concurrently with, and/or subsequently to step (a), culturing said somatic or somatic cell nucleus cell in the presence of one or more DNA methyltransferase inhibitors. The one or more DNA methyltransferase inhibitors may be selected from the group consisting of: 5-azacytidine (5-aza-CR), 5-aza-2′-deoxycytidine (5-aza-CdR), zebularine, procaine, (−)-epigallocatechin-3-gallate (EGCG), RG108, and an antisense RNA targeting a DNA methyltransferase gene expressed by said somatic cell or somatic cell nucleus.

The method may further comprise prior to, concurrently with, and/or subsequently to step (a), culturing said somatic cell or somatic cell nucleus in the presence of one or more Histone DeAcetylase (HDAC) inhibitors. The one or more HDAC inhibitors may be selected from the group consisting of: Sodium Butyrate, Trichostatin A, hydroxamic acids, cyclic tetrapeptides, trapoxin B, depsipeptides, benzamides, electrophilic ketones, aliphatic acid compounds, phenylbutyrate, valproic acid, hydroxamic acids, vorinostat (SAHA), belinostat (PXD101), LAQ824, panobinostat (LBH589), entinostat (MS275), C1994, mocetinostat (MGCD0103), and antisense RNA targeting an HDAC gene expressed by said somatic cell or somatic cell nucleus.

Step (a) in the method may further comprise permeabilizing said somatic cell or somatic cell nucleus.

Step (a) in the method may further comprise introducing said one or more reprogramming agents into said somatic cell or somatic cell nucleus by one or more methods selected from the group consisting of: microinjection, electroporation, fusion with a liposome, fusion with an enucleated donor cell, fusion or contact with a cytoplasmic bleb containing at least one reprogramming factor, and culturing said somatic cell or somatic cell nucleus in a medium containing said reprogramming composition and optionally containing a cell and/or nucleus entry agent. The cell and/or nucleus entry agent may be selected from the group consisting of Streptolysin O, digitonin, a cationic amphiphile, and any combination thereof.

At least one of said reprogramming agents may be provided by a source cell that secretes at least one reprogramming agent into an aqueous medium containing said somatic cell or somatic cell nucleus and/or at least one of said reprogramming agents may be provided by a cell extract obtained from a pluripotent cell.

Step (a) in the method may further comprise contacting the somatic cell or somatic cell nucleus with the reprogramming composition more than once and/or for a prolonged incubation period.

The method may further comprise phenotypic monitoring of said somatic cell or somatic cell nucleus to measure its adoption of one or more phenotypes associated with the target cell or nucleus. Exemplary phenotypic monitoring may comprise one or more measurements selected from the group consisting of detecting the expression of one or more genes associated with the target cell type, detecting the methylation state and/or histone acetylation state of the promoters of one or more genes associated with the target cell type, measuring the expression of an engineered reporter construct that may be differentially active between said somatic cell and said target cell type, and detecting morphology indicative of said target cell type. The engineered cell reporter construct may comprise a gene encoding a marker gene coupled to a muscle-specific promoter. The marker gene may encode a fluorescent protein which optionally may be a green, blue, cyan, or yellow fluorescent protein. The marker gene may encode a selectable marker. The muscle-specific promoter may comprise a Myogenin, MyoD or Troponin T promoter sequence, or may comprise a slow/cardiac troponin C (cTnC) promoter sequence, smooth muscle myosin heavy chain promoter sequence, smooth-muscle alpha-actin promoter sequence, and SM22alpha promoter sequence, one or more CArG [CC(A}Trich)6GG] boxes, SRF binding site, or GATA-4 binding site.

The reprogramming composition may comprise a reprogramming polypeptide. The reprogramming polypeptide may be essentially free from a polynucleotide that encodes said reprogramming polypeptide. The reprogramming polypeptide may comprise at least one protein transduction domain that facilitates entry of the reprogramming polypeptide into the somatic cell or somatic cell nucleus, which may be independently selected from the group consisting of any of the polypeptides of SEQ ID NO: 1 through 10. The reprogramming composition may be essentially free from viruses capable of genetically modifying said somatic cell or somatic cell nucleus, transfection media, Streptolysin O, digitonin, cationic amphiphiles, liposomes, and other constituents that would facilitate entry of a polynucleotide into said somatic cell or somatic cell nucleus.

The reprogramming composition may comprise one or more reprogramming polypeptides selected from the group consisting of: MyoD, IGF-1, aFGF, Activin A, or may comprise one or more reprogramming agents selected from the group consisting of Activin A, aFGF, Akt1, Akt2, Bmp4, Capsulin, c-MET, one or more E-proteins, E12, E47, Eya2, FoxO3, Gli, HEB, Hepatocyte Growth Factor (HGF) or Scatter factor (SF), an antagonist of Id, IGF-1, Lbx1, an antagonist of Mdfi, MEF2, Mef2c, Mef2d, MEF3, Meis, Meox2, microRNA-1, microRNA-1-1, microRNA-1-2, microRNA-27, microRNA-133, microRNA-133a-1, microRNA-133a-2, microRNA-133b, microRNA-206, microRNA-208, microRNA-208b, microRNA-499, MRF4, My5, Myf5, Myh3, Myh6, Myh7, Myh7b, MyoD, Myogenin, MyoR, p38, Paraxis, Pax3, Pax7, Pbx, Pitx2, an antagonist of Pur-beta, Six1, Six4, Sonic Hedgehog, an antagonist of Hedgehog signal transduction or the Gli transcription factor, an antagonist of Sox6, an antagonist of Sp3, Sp1, TAF3, Tbx1, TRF3, Wnt, and Wnt ligand.

The present disclosure further provides methods of identifying a reprogramming agent that converts or enhances or potentiates the conversion of a somatic cell or somatic cell nucleus into a target type, wherein said target type is skeletal muscle, cardiac muscle, or smooth muscle. The method may comprise: (a) providing a candidate reprogramming composition from which a candidate reprogramming agent has been depleted, omitted, or otherwise excluded and a reprogramming composition that contains said candidate reprogramming agent; (b) culturing a somatic cell or somatic cell nucleus that is not of the target cell type in the presence of said candidate reprogramming composition, and culturing a control somatic cell or control somatic cell nucleus in the presence of said reprogramming composition; and (c) monitoring said somatic cell or somatic cell nucleus to measure its adoption of one or more phenotypes associated with the target cell type, and identifying said candidate reprogramming agent as a reprogramming agent if said adoption of one or more phenotypes associated with the target cell type is significantly decreased or delayed relative to said control cell or control cell nucleus. Alternatively, step (a) may comprise providing a candidate reprogramming composition comprising a fraction or mixture of fractions of a reprogramming composition, wherein said reprogramming composition comprises conditioned medium or a cell or nuclear extract, and a control reprogramming composition that lacks said one or more of said fractions; and/or step (b) may comprise culturing a somatic cell or somatic cell nucleus that is not of the target cell type in the presence of said candidate reprogramming composition, and culturing a control somatic cell or control somatic cell nucleus in the presence of said control reprogramming composition; and/or step (c) may comprise monitoring said somatic cell or somatic cell nucleus to measure its adoption of one or more phenotypes associated with the target cell type, and identifying said one or more candidate reprogramming agents as reprogramming agents if said adoption of one or more phenotypes associated with the target cell type is significantly increased or accelerated relative to said control cell or control cell nucleus cultured. The target cell type may be skeletal muscle.

The reprogramming composition may be able to convert a cell or nucleus of said somatic cell type into the target cell type. The reprogramming composition may be an extract obtained from a muscle cell or myoblast cell, or said reprogramming composition may be a muscle cell or myoblast cell conditioned medium. The extract may be a whole cell extract, nuclear extract, or cytoplasmic extract. The extract may be obtained from the same type of cell as the target cell type. The candidate reprogramming agent may be identified by comparison of gene expression of cells of the target type to a control cell of a different type. The control cell may be selected from the group consisting of: a fibroblast, kidney cell, embryonic stem cell, mesenchymal stem cell, somatic cells other than the target cell type, and any combination thereof.

In the context of methods for identifying reprogramming agents or evaluating the ability of a candidate reprogramming agent to effect reprogramming, a control cell may be utilized. Most typically the control cell will be of the same type as the treated cell (a cell treated with a candidate reprogramming composition or candidate reprogramming agent), however in some instances a control cell may be of a different type. A control cell is typically treated differently than the treated cell, e.g., left untreated or treated with a composition and/or under conditions that differ in some respect from the composition with which the treated cell is treated. For example, in embodiments utilizing cell extracts as reprogramming compositions, a control extract taken from a different cell type may be used. As another example, a factor may be added to a candidate reprogramming composition and/or caused to be expressed by the extract with which the treated cell is treated, but such factor may be omitted from a control extract. As a further example, a treated cell may be treated with a combination of candidate reprogramming agents, and a control cell may be treated with more or fewer candidate reprogramming agents. Exemplary additional components or omitted components include any of the reprogramming agents and candidate reprogramming agents identified herein, including without limitation: polypeptides and polynucleotides, cytoskeletal disruptors, chromatin remodeling agents, transcription modifiers, small molecules, short interfering RNAs or analogs, cell and/or nucleus entry agents, etc. Additionally, a control cell may be treated with the same candidate reprogramming composition as the treated cell but with differences in the time course of treatment (e.g., treatment duration or number of treatments) or under different culture conditions (e.g., degree of confluence, medium, temperature, atmospheric condition including concentration of oxygen, carbon dioxide, humidity, pressure, etc.). Such methods may permit evaluation of whether an additional component or omitted component of a candidate reprogramming composition, or any other difference in treatment condition, converts or enhances or potentiates the conversion of a somatic cell or somatic cell nucleus into a target type.

Phenotypic monitoring may comprise one or more measurements selected from the group consisting of detecting the expression of one or more genes associated with the target cell type, detecting the methylation state and/or histone acetylation state of the promoters of one or more genes associated with the target cell type, measuring the expression of an engineered reporter construct that may be differentially active between said somatic cell and said target cell type, and detecting morphology indicative of said target cell type. The engineered reporter construct may comprise a gene encoding a marker gene coupled to a muscle-specific promoter. The marker gene may encode a fluorescent protein which optionally may be a green, blue, cyan, or yellow fluorescent protein. The marker gene may encode a selectable marker. The muscle-specific promoter may comprise a Myogenin, MyoD or Troponin T promoter sequence. The muscle-specific promoter may comprise slow/cardiac troponin C (cTnC) promoter sequence, smooth muscle myosin heavy chain promoter sequence, smooth-muscle alpha-actin promoter sequence, and SM22alpha promoter sequence, one or more CArG [CC(A}Trich)6GG] boxes, SRF binding site, or GATA-4 binding site.

The present disclosure provides further methods of identifying a reprogramming agent that converts or enhances or potentiates the conversion of a somatic cell or somatic cell nucleus into a target type, wherein said target type is skeletal muscle, cardiac muscle, or smooth muscle. The method may comprise: (a) providing a candidate reprogramming composition comprising multiple candidate reprogramming agents; (b) culturing a somatic cell or somatic cell nucleus that is not of the target cell type in the presence of said candidate reprogramming composition and culturing a control somatic cell or control somatic cell nucleus in the absence of said candidate reprogramming composition; and (c) monitoring one or more phenotypes of said somatic cell or somatic cell nucleus to measure its adoption of one or more phenotypes associated with the target cell type, and identifying said candidate reprogramming composition as a reprogramming composition if said adoption of one or more phenotypes associated with the target cell type is significantly increased or accelerated relative to control cells or control cell nuclei cultured in the absence of said candidate reprogramming composition. Alternatively, the method may comprise (a) causing a somatic cell or somatic cell nucleus that is not of the target cell type to express multiple candidate reprogramming agents, and culturing a control somatic cell or control somatic cell nucleus without causing the expression of one or more of said candidate reprogramming genes; and (b) monitoring said somatic cell or somatic cell nucleus to measure its adoption of one or more phenotypes associated with the target cell type, and identifying said candidate reprogramming genes as reprogramming genes if said adoption of one or more phenotypes associated with the target cell type is significantly increased or accelerated relative to said control cell or control cell nucleus. The target cell type may be skeletal muscle.

Said multiple candidate reprogramming agents may comprise one or more genes selected from the group consisting of: MyoD, IGF-1, aFGF, and Activin A, or may comprise one or more substances selected from the group consisting of: Activin A, aFGF, Akt1, Akt2, Bmp4, Capsulin, c-MET, one or more E-proteins, E12, E47, Eya2, FoxO3, Gli, HEB, Hepatocyte Growth Factor (HGF) or Scatter factor (SF), an antagonist of Id, IGF-1, Lbx1, an antagonist of Mdfi, MEF2, Mef2c, Mef2d, MEF3, Meis, Meox2, microRNA-1, microRNA-1-1, microRNA-1-2, microRNA-27, microRNA-133, microRNA-133a-1, microRNA-133a-2, microRNA-133b, microRNA-206, microRNA-208, microRNA-208b, microRNA-499, MRF4, My5, Myf5, Myh3, Myh6, Myh7, Myh7b, MyoD, Myogenin, MyoR, p38, Paraxis, Pax3, Pax7, Pbx, Pitx2, an antagonist of Pur-beta, Six1, Six4, Sonic Hedgehog, an antagonist of Hedgehog signal transduction or the Gli transcription factor, an antagonist of Sox6, an antagonist of Sp3, Sp1, TAF3, Tbx1, TRF3, Wnt, Wnt ligand, and any combination thereof. Said multiple candidate reprogramming agents may comprise one or more polypeptides having increased expression in skeletal muscle cells or myoblast cells relative to a control cell of a different type. The control cell may be selected from the group consisting of: a fibroblast, kidney cell, embryonic stem cell, mesenchymal stem cell, somatic cells other the target cell type, and any combination thereof.

In another embodiment, the present disclosure provides composition comprising one or more reprogramming agents identified by any of the foregoing methods, which may optionally be in an amount sufficient to convert a somatic cell or cell nucleus to a target cell type and a somatic cell or somatic cell nucleus that is not of the target cell type, wherein said target cell type may be skeletal muscle, cardiac muscle, or smooth muscle.

In another embodiment, the present disclosure provides a cell therapy method which may comprise administration to a subject in need thereof cells such as skeletal muscle, cardiac muscle, or smooth muscle cells produced according to the method of any of the foregoing methods. Exemplary cell therapy methods may be used to prevent or treat a disease or condition selected from the group consisting of diseases and dysfunctions involving skeletal muscle, myopathy, chronic fatigue syndrome, fibromyalgia, ALS (Lou Gehrig's disease), MS (multiple sclerosis), Central Fibrillar Shy-Magee Syndrome, Thomsen disease, Statin-associated myopathy, muscular dystrophy (including Duchenne, Becker, limb girdle, congenital, facioscapulohumeral, myotonic, oculopharyngeal, distal, and Emery-Dreifuss), spinal muscular atrophy, Brown-Vialetto-Van Laere syndrome, Fazio-Londe (FL) syndrome, inclusion body myositis, polymyalgia rheumatica, dermatomyositis, polymyositis, rhabdomyolysis, compartment syndrome, muscle atrophy, nemaline myopathy, multi/minicore myopathy, centronuclear myopathy (or myotubular myopathy), mitochondrial myopathies, familial periodic paralysis, inflammatory myopathies, metabolic myopathies, glycogen storage diseases, lipid storage disorder, myositis ossificans, myoglobinurias, alcoholic myopathy, and trauma.

Additional exemplary cell therapy methods may be used to prevent or treat a disease or condition selected from the group consisting of: diseases and dysfunctions involving cardiac muscle, cardiomyopathy, intrinsic cardiomyopathies, extrinsic cardiomyopathies, genetic cardiomyopathies, acquired cardiomyopathies, mixed cardiomyopathies, metabolic/storage cardiomyopathies, inflammatory cardiomyopathies, endocrine cardiomyopathies, toxicity cardiomyopathies, neuromuscular cardiomyopathies, nutritional cardiomyopathies, ischemic cardiomyopathies, hypertrophic cardiomyopathy, arrhythmogenic right ventricular cardiomyopathy, isolated ventricular non-compaction, mitochondrial myopathy, dilated cardiomyopathy, restrictive cardiomyopathy, Takotsubo cardiomyopathy, Loeffler endocarditis, amyloidosis, cardiomyopathy associated with hemochromatosis, Chagas disease, diabetic cardiomyopathy, cardiomyopathy associated with hyperthyroidism, cardiomyopathy associated with chemotherapy, alcoholic cardiomyopathy, muscular dystrophy, and ischemic cardiomyopathy.

Further exemplary cell therapy methods may be used to prevent or treat a diseases or condition selected from the group consisting of: diseases involving smooth muscle, vascular diseases, atherosclerosis, arteriosclerosis, arteriolosclerosis, atherosclerosis, stenosis, aneurysm, claudication, infarction, cirrhosis, fibrosis of the lung, asthma, allergies, and generalized smooth-muscle disease.

Yet further exemplary cell therapy methods may be used to prevent or treat a diseases or condition selected from the group consisting of: muscle atrophy, cachexia, anorexia, Dejerine Sottas syndrome, inactivity, bed rest, limb or joint immobilization, congestive heart failure, chronic obstructive pulmonary disease, liver disease, starvation, aging, and muscle atrophy resulting from microgravity or weightlessness.

### BRIEF DESCRIPTION OF THE DRAWINGS

FIG. 1. The C2C12 Muscle Differentiation System. C2C12 cells were cultured in 20% serum till 90% confluent and then either fixed (A. t=0) or switched to 2% serum and cultured for an additional 6 days (B. t=6 days). Cells were stained for nuclei (Dapi: blue), actin (with Rhodamine-phalloidin: red) and myosin (immunofluorescence with an anti-myosin antibody: green), as indicated. Bottom right panels depict merged views of all three stains.

FIG. 2. Myotube formation in 2% serum conditions (higher magnification view). C2C12 cells were cultured in 20% serum till 90% confluent and then either fixed (A. t=0) or switched to 2% serum and cultured for an additional 6 days (B. t=6 days). Shown are merged views of cells stained for nuclei (Dapi: blue) and myosin (immunofluorescence with an anti-myosin antibody: green); actin (with Rhodamine-phalloidin: red) and myosin; or, nuclei, myosin and actin together (right panels), as indicated.

FIG. 3A. Differentiation of C2C12 myoblasts: expression of MyoD. C2C12 cells were cultured in 20% serum till 85% confluent and then either fixed (t=0) or switched to 2% serum and cultured for an additional 3 days (t=3). Cells were stained for nuclei (Dapi: blue) and the transcription factor MyoD (immunofluorescence with an anti-MyoD antibody: green), as indicated.

FIG. 3B. Differentiation of C2C12 myoblasts: expression of Myogenin. C2C12 cells were cultured in 20% serum till 85% confluent and then either fixed (t=0) or switched to 2% serum and cultured for an additional 3 days (t=3). Cells were stained for nuclei (Dapi: blue) and the transcription factor Myogenin (immunofluorescence with an anti-Myogenin antibody: green), as indicated.

FIG. 3C. Differentiation of C2C12 myoblasts: expression of Troponin T. C2C12 cells were cultured in 20% serum till 85% confluent and then either fixed (t=0) or switched to 2% serum and cultured for an additional 3 days (t=3). Cells were stained for nuclei (Dapi: blue) and Troponin T (immunofluorescence with an anti-Troponin T antibody: green), as indicated.

FIG. 4A. Protein Profile in C2C12 extracts: RIPA lysis vs Sonication. C2C12 cells were harvested at different time points through differentiation (as indicated) and lysed either in the detergent containing RIPA buffer or by Sonication/Freeze-thaw. The lysates were resolved by SDS-PAGE and examined by Coomassie blue staining. The position of Molecular weight markers is indicated on the left in kilodaltons.

FIG. 4B. Immunoblot detection of proteins in extracts of differentiating C2C12 cells. C2C12 cells were harvested at different time points through differentiation (as indicated) as were control C166 cells. Equal amounts of total protein of each lysate were resolved by SDS-PAGE and immunoblotted with antibodies against Myogenin, MyoD, Troponin T or JunB, as indicated on the right. The position of Molecular weight markers is indicated on the left in kilodaltons.

FIG. 5. Detection of Muscle specific gene expression in extracts of differentiating C2C12 cells by RT-PCR Total RNA prepared from C2C12 cells at different stages of differentiation (t=0, t=3, t=6) or from 293T cells or HeLa cells were used in an RT-PCR reaction with primers specific to the muscle specific genes, MyoD, Myogenin, as indicated on the right. Products of the reactions were resolved by DNA-agarose gel electrophoresis and detected by virtue to the fluorescent dye Ethidium Bromide, bound to DNA.

FIG. 6. Survival and Uptake of Rhodamine-Albumin by permeabilized 293T cells in the presence of differentiating C2C12 Extracts (t=3, Day 4). Cells were incubated with Rhodamine-labeled Albumin and C2C12 cell extracts during SLO mediated permeabilization. Images shown were taken four days post treatment. Left panels: bright field microscopy image; Right panels: fluorescence microscopy view of the same field. Magnification: 40×.

FIG. 7. Detection of Muscle specific MyoD RNA in NHDF cells incubated with extracts of differentiating C2C12 cells by RT-PCR. Permeabilized primary neonatal Normal Human Dermal Fibroblast (NHDF-N) cells were incubated with either NHDF cell extract (“self” control) or extracts of C2C12 cells collected at different stages of differentiation (at time zero (T0), time=3 days (T3) or time=6 days (T6), post induction of differentiation). Total RNA was collected at different times post permeabilization and analyzed by RT-PCR. The same primers were also used with total RNA prepared from unpermeabilized NHDF cells (NHDF-N) or C2C12 cells, as indicated. Products of the reactions were resolved by DNA-agarose gel electrophoresis and detected by virtue of the fluorescent dye Ethidium Bromide, bound to DNA. Upper panel: RT-PCR with primers specific to the ubiquitously expressed house keeping gene GAPDH. Lower panel: RT-PCR with increasing amounts of RNA from the permeabilized NHDF cells, from unpermeabilized NHDF cells (NHDF-N) or C2C12 cells (as indicated) and primers specific to the muscle specific gene, MyoD. The positions of Molecular weight markers (base pairs) are indicated on the left.

FIG. 8. Detection of Muscle specific MyoD RNA in 293T cells incubated with extracts of differentiating C2C12 cells by RT-PCR. Permeabilized 293T cells were incubated with either 293T cell extract (“self” control) or extracts of C2C12 cells collected at different days post induction of differentiation (at time zero (T0), time=3 days (T3) or time=6 days (T6)). Total RNA was collected at different times post permeabilization and analyzed by RT-PCR. The same primers were also used with total RNA prepared from unpermeabilized 293T or C2C12 cells or with “no mRNA”, as indicated. Upper panel: RT-PCR with primers to GAPDH. Lower panel: RT-PCR with primers specific to the muscle specific gene, MyoD.

FIG. 9. Detection of Muscle specific MyoD RNA in 293T cells treated with transcription modifying agents post incubation with extracts of differentiating C2C12 cells. (A) 293T cells were permeabilized and incubated with either 293T cell extract (“self” control) or extracts of C2C12 cells (C2C12). The cells were then left untreated (un) or treated with 5 Azacytidine (10 uM) or Sodium Butyrate (10 mM) or Trichostatin A (10 nM) or 5 Azacytidine (10 uM) and Sodium Butyrate (10 mM) or 5 Azacytidine (10 uM) and Trichostatin A (10 nM), as indicated. Total RNA was collected at different times post permeabilization and analyzed by RT-PCR with primers specific to the muscle specific gene, MyoD (upper panel) or to GAPDH (lower panel). (B) The same primers were also used with total RNA prepared from unpermeabilized 293T or C2C12 cells or with “no mRNA”, as indicated.

FIG. 10. Detection of Muscle specific MyoD RNA in 293T cells treated with transcription modifying agents prior to permeabilization and incubation with extracts of differentiating C2C12 cells. 293T cells were left untreated (un) or treated with 5 Azacytidine (10 uM) or Sodium Butyrate (10 mM) or Trichostatin A (10 nM) or 5 Azacytidine (10 uM) and Sodium Butyrate (10 mM) or 5 Azacytidine (10 uM) and Trichostatin A (10 nM) for 48 hours, as indicated. The cells were then incubated with either 293T cell extract (“self” control) or extracts of C2C12 cells (C2C12). Total RNA was collected at different times post permeabilization and analyzed by RT-PCR. The same primers were also used with total RNA prepared from unpermeabilized 293T or C2C12 cells or with “no mRNA”, as indicated. Upper panel: RT-PCR reactions performed with primers specific to the muscle specific gene, MyoD. Lower panel: RT-PCR reactions performed with primers specific to GAPDH.

FIG. 11A. Detection of Muscle specific MyoD and Myogenin RNA in 293T cells treated with transcription modifying agents prior to incubation with extracts of differentiating C2C12 cells. 293T cells were left untreated (un) or treated with 5 Azacytidine (10 uM) or Sodium Butyrate (10 mM) or Trichostatin A (10 nM) or 5 Azacytidine (10 uM) and Sodium Butyrate (10 mM) or 5 Azacytidine (10 uM) and Trichostatin A (10 nM) for 48 hours, as indicated. The cells were then primed with either 293T cell extract (“self” control) or extracts of C2C12 cells (C2C12). Total RNA was collected at different times post permeabilization (as indicated) and analyzed by RT-PCR using different primer sets. The same primers were also used with total RNA prepared from unpermeabilized 293T or C2C12 cells or with “no mRNA”, as indicated. Top panel: RT-PCR reactions performed with primers specific to the muscle specific gene, MyoD. Middle panel: RT-PCR reactions performed with primers specific to the muscle specific gene, Myogenin. Lower panel: RT-PCR reactions performed with primers specific to GAPDH.

FIG. 11B. Detection of Muscle specific Myosin Heavy Chain and Troponin T RNA in 293T cells treated with transcription modifying agents prior to permeabilization and incubation with extracts of differentiating C2C12 cells. Total RNA from samples of the experiment described in FIG. 13A was analyzed by RT-PCR with primers specific to the muscle specific genes, Myosin Heavy Chain, MHC (upper panel) or Troponin T, TnT (lower panel).

FIG. 12. Detection of Muscle specific MyoD RNA in 293T cells treated with C2C12 conditioned media post permeabilization. 293T cells were permeabilized and primed with either 293T cell extract (“self” control) or extracts of differentiating C2C12 cells (T0). Immediately post incubation, the cells were allowed to recover in either 100% regular media (C2C12 media with reduced (5%) serum), 70% regular media supplemented with 30% C2C12 conditioned media (CM) or 100% C2C12 conditioned media, as indicated. Total RNA was collected at different times post permeabilization and analyzed by RT-PCR. Upper panel: RT-PCR reactions performed with primers specific to the muscle specific gene, MyoD. Lower panel: RT-PCR reactions performed with primers specific to GAPDH.

FIG. 13. Detection of Muscle specific MyoD and Myogenin RNA in NIH3T3 cells by RT-PCR. NIH 3T3 cells were left untreated (Un) or treated with 5 azacytidine (10 uM), Trichostatin A (10 nM) or a combination of 5 azacytidine and TSA for 48 hours, as indicated. The cells were then permeabilized and incubated with either NIH3T3 cell extract (“self” control) or extracts of C2C12 cells (T0). Immediately post incubation, the cells were allowed to recover in the absence (−post) or presence (+post) of post treatments (50% C2C12 conditioned media (CM) plus a continuation of respective pre-treatments). Total RNA was collected at different times post priming and analyzed by RT-PCR with primers specific for GAPDH or the muscle specific genes, MyoD or Myogenin. The same primers were also used with total RNA prepared from unpermeabilized NIH3T3 or C2C12 cells or with “no RNA”, as indicated. Upper panel: RT-PCR reactions performed with primers specific to the muscle specific gene, MyoD. Middle panel: RT-PCR reactions performed with primers specific to the muscle specific gene, Myogenin. Lower panel: RT-PCR reactions performed with primers specific to GAPDH.

FIG. 14. Prolonged detection of Muscle specific MyoD RNA in NIH3T3 cells treated with transcription modifying agents, C2C12 conditioned media and specific muscle-inducing factors. NIH 3T3 cells were left untreated (−pre) or treated (+pre) with a combination of 5 azacytidine (10 uM) and TSA (10 nM) for 48 hours, as indicated. The cells were then permeabilized and incubated with either NIH3T3 cell extract (“self”) or extracts of C2C12 cells (T0). Post incubation, the cells were allowed to recover in the absence (Un) or presence (Treated) of post treatments (50% conditioned media with 5-aza and TSA (CM+A+T) with added IGF-1, aFGF and Activin A). Total RNA was collected at different times post priming and analyzed by RT-PCR with primers specific for GAPDH (lower panel) or the muscle specific genes, MyoD (upper panel).

### DETAILED DESCRIPTION

Growing evidence for the plasticity of cell fate has opened the possibility of reprogramming of somatic cells in the laboratory. Reprogramming refers to the conversion of one somatic cell type into another, a process that entails the reinstruction of the gene expression profile of a cell. Reprogrammed somatic cells may be used for cell or tissue therapy such that a patient's own cells or histocompatible cells can be used for treatment of disease or injury.

Each somatic cell type expresses a characteristic repertoire of genes, which may be regulated by environmental cues or factors that cause and/or maintain a particular programmed state through signaling networks that lead to the expression and/or activation/repression of regulatory transcription factors and to epigenetic modifications in DNA/chromatin conformation that determine whether a gene is transcribed or not. By manipulating one or more of these regulatory elements, a cell can be caused to adopt a new differentiated, or reprogrammed, state. Without intent to be limited by theory, Applicants hypothesized that somatic cells of a given type contain key regulatory elements which can be sufficient to reprogram another cell to become a cell of that type. Thus, a recipient cell may be reprogrammed by exposure to reprogramming agents, which include donor cell extract, or one or more endogenous or recombinant reprogramming factors or functional fragments, variants or fusion polypeptides or cell extracts which contain said endogenous reprogramming factors, polynucleotides encoding reprogramming agents, small molecule activators or regulators or mimics of reprogramming agents, or antagonists of inhibitors of reprogramming agents (e.g., using RNAi mediators such as short interfering RNAs or analogs thereof). In exemplary embodiments, the reprogramming factors include endogenous or recombinant reprogramming polypeptides or functional fragments, variants or fusion polypeptides containing, which e.g., may be comprised in a donor cell cytoplasm, may be synthesized or recombinant, may optionally include one or more modifications, and may optionally be purified. These factors may be introduced into the recipient cell by a variety of methods known in the art, which include by way of example electroporation, microinjection, liposomes, cationic lipids, cell permeabilization, incubation or contacting with donor cell cytoplasm or cytoplasmic blebs and/or linkage thereof to one or more protein transduction domains (PTD) or nuclear translocation domain or nuclear localization signals moieties (NTD or NTM or NTS moieties).

Reprogramming efficiency may be improved by treatment of the recipient cells with cytoskeletal disruptors, chromatin remodeling agents, and/or transcription modifiers, which may occur before, during, and/or after contact with reprogramming factors.

Reprogramming compositions according to the present disclosure may comprise one or more of the following compounds or agonists, mimics, analogs, active domains, or equivalents thereof: Activin A, aFGF, Akt1, Akt2, Bmp4, Capsulin, c-MET, one or more E-proteins, E12, E47, Eya2, FoxO3, Gli, HEB, Hepatocyte Growth Factor (HGF) or Scatter factor (SF), an antagonist of Id, IGF-1, Lbx1, an antagonist of Mdfi, MEF2, Mef2c, Mef2d, MEF3, Meis, Meox2, microRNA-1, microRNA-1-1, microRNA-1-2, microRNA-27, microRNA-133, microRNA-133a-1, microRNA-133a-2, microRNA-133b, microRNA-206, microRNA-208, microRNA-208b, microRNA-499, MRF4, My5, Myf5, Myh3, Myh6, Myh7, Myh7b, MyoD, Myogenin, MyoR, p38, Paraxis, Pax3, Pax7, Pbx, Pitx2, an antagonist of Pur-beta, Six1, Six4, Sonic Hedgehog, an agonist of Hedgehog signal transduction or the Gli transcription factor, an antagonist of Sox6, an antagonist of Sp3, Sp1, TAF3, Tbx1, TRF3, Wnt, or a Wnt ligand. See Bismuth et al., Genetic regulation of skeletal muscle development, Exp Cell Res. 2010 Nov. 1; 316(18):3081-6; Wang et al., Development. 2001 November; 128(22):4623-33; Peng et al., Genes Dev. 2003 Jun. 1; 17(11):1352-65; Rudnicki et al., Cell. 1993 Dec. 31; 75(7):1351-9; Carvajal et al., Exp Cell Res. 2010 Nov. 1; 316(18):3014-8; Tapscott, Development. 2005 June; 132(12):2685-95.

In another aspect, the present disclosure provides methods for identifying reprogramming agents. Active materials present in donor cytoplasm and/or conditioned media that contribute to reprogramming may be identified by fractionation, subtractive hybridization, or comparison of gene expression profiles (which can be determined using microarrays and other techniques known in the art). Candidate active materials may be tested by selective depletion (e.g., using immunodepletion, RNAi, genetic alteration of the source cell, and other methods known in the art), where reduction in reprogramming activity would provide evidence that the candidate reprogramming agent(s) contribute to reprogramming activity; further evidence of reprogramming activity could be obtained in a “rescue” experiment in which a purified or recombinant agent can restore reprogramming activity of a selectively depleted preparation lacking said agent. Candidate active material may also be tested by determining increasing the amount of such agent in a reprogramming cell extract and/or conditioned medium can enhance reprogramming activity. Purified preparations comprising one or more candidate reprogramming agents may be added to cells to identify an agent or agents that are sufficient for reprogramming. In another aspect, the present disclosure provides a reprogramming composition comprising one or more reprogramming agents. In a further aspect, the present disclosure provides a composition comprising recipient cells and exogenously added reprogramming agents which may be in an amount sufficient to effect reprogramming of said recipient cells.

Methods for introducing reprogramming agents into cells or using same to reprogram cell nuclei

Numerous methods are known to one of skill in the art for effecting transport and delivery of a desired polypeptides or nucleic acids or small molecules into a recipient cell or cell nucleus and may be used to effectively deliver reprogramming agents into cells or cell nuclei These methods include by way of example electroporation, microinjection, liposomes, cationic lipids, cell permeabilization, incubation or contacting with donor cell cytoplasm or cytoplasmic blebs and/or linkage thereof to one or more protein transduction domains (PTD) or nuclear translocation domain or nuclear localization signals moieties (NTD or NTM or NTS moieties). Examples of such moieties which facilitate nuclear delivery of substituents attached thereto include by way of example SV40 T-antigen localization signal, the C-terminus of apoptin, acridine nuclear localization signal, polyargine (arg11), s4 13-PV, adenovirus hexon protein, PV-S4(13), RR-S4(13), et al. NLSs are generally short, positively charged (basic) domains that serve to direct the moiety to which they are attached to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-.beta. nuclear localization signal (ARRRRP); NF kappa B p50 (EEVQRKRQKL; Ghosh et al., Cell 62:1019 (1990); NF kappa B p65 (EEKRKRTYE; Nolan et al, Cell 64:961 (1991); and others (see, for example, Boulikas, J. Cell. Biochem. 55(1):32-58 (1994), hereby incorporated by reference) and double basic NLS's exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann. Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990.

For example, electroporation may be used to introduce DNA into mammalian cells (Neumann, E. et al. (1982) EMBO J. 1, 841-845), as well as plant and bacterial cells, and may also be used to introduce proteins (Marrero, M. B. et al. (1995) J. Biol. Chem. 270, 15734-15738; Nolkrantz, K. et al. (2002) Anal. Chem. 74, 4300-4305; Rui, M. et al. (2002) Life Sci. 71, 1771-1778). Cells (such as the cells of this disclosure) can be suspended in a buffered solution containing the protein, DNA, or other molecule of interest are placed in a pulsed electrical field. Briefly, high-voltage electric pulses result in the formation of small (nanometer-sized) pores in the cell membrane. Molecules enter the cell via these small pores or during the process of membrane reorganization as the pores close and the cell returns to its normal state. The efficiency of delivery is dependent upon the strength of the applied electrical field, the length of the pulses, temperature and the composition of the buffered medium. Electroporation is successful with a variety of cell types, even some cell lines that are resistant to other delivery methods, although the overall efficiency is often quite low. Some cell lines remain refractory even to electroporation unless partially activated.

Microinjection can be used to introduce femtoliter volumes containing molecules of interest directly into the nucleus of a cell. It has been used to introduce DNA directly into the nucleus of a cell (Capecchi, M. R. (1980) Cell 22, 470-488) where it was integrated directly into the host cell genome, thus creating an established cell line bearing the sequence of interest. Proteins such as antibodies (Abarzua, P. et al. (1995) Cancer Res. 55, 3490-3494; Theiss, C. and Meller, K. (2002) Exp. Cell Res. 281, 197-204) and mutant proteins (Naryanan, A. et al. (2003) J. Cell Sci. 116, 177-186) can also be directly delivered into cells via microinjection. Other molecules of interest, including RNA, episomal DNA, small molecules, proteins, etc., can also be introduced into cells by similar methods. Microinjection has the advantage of introducing macromolecules directly into the cell, thereby bypassing exposure to potentially undesirable cellular compartments such as low-pH endosomes. Microinjection can be performed manually and using semi-automated and fully automated microinjection systems, e.g., as described in: Matsuoka et al., Journal of Biotechnology, Volume 116, Issue 2, 16 Mar. 2005, Pages 185-194; Zhang and Yu, Current Opinion in Biotechnology, Volume 19, Issue 5, October 2008, Pages 506-510; Wang et al., PLoS One. 2007 Sep. 12; 2(9):e862; Ito et al., U.S. PGPub. No. 2008/0299647; Ito et al., U.S. PGPub. No. 2008/0268540; Japanese Patent No. 2624719; Ando et al., U.S. PGPub. No. 2008/0002868; Myiawaki et al., U.S. PGPub. No. 2007/0087436.

Liposomes can also be used to introduce molecules into cells. Liposomes have been used to deliver oligonucleotides, DNA (gene) constructs and small drug molecules into cells (Zabner, J. et al. (1995) J. Biol. Chem. 270, 18997-19007; Felgner, P. L. et al. (1987) Proc. Natl. Acad. Sci. USA 84, 7413-7417). Certain lipids, when placed in an aqueous solution and sonicated, form closed vesicles consisting of a circularized lipid bilayer surrounding an aqueous compartment. These vesicles or liposomes can be formed in a solution containing the molecule to be delivered. In addition to encapsulating DNA in an aqueous solution, cationic liposomes can spontaneously and efficiently form complexes with DNA, with the positively charged head groups on the lipids interacting with the negatively charged backbone of the DNA. The exact composition and/or mixture of cationic lipids used can be altered, depending upon the macromolecule of interest and the cell type used (Felgner, J. H. et al. (1994) J. Biol. Chem. 269, 2550-2561). The cationic liposome strategy has also been applied successfully to protein delivery (Zelphati, O. et al. (2001) J. Biol. Chem. 276, 35103-35110). Because proteins are more heterogeneous than DNA, the physical characteristics of the protein such as its charge and hydrophobicity will influence the extent of its interaction with the cationic lipids.

Cationic lipid complexes can also be used to introduce molecules into cells. For example, the Pro-Ject Protein Transfection Reagent may be used. The Pro-Ject Protein Transfection Reagent utilizes a cationic lipid formulation that is noncytotoxic and is capable of delivering a variety of proteins into numerous cell types. The molecule to be introduced is mixed with the liposome reagent and is overlayed onto cultured cells. The liposome:molecule complex is believed to facilitate entry into cells via fusion with the cell membrane or internalization via an endosome. The molecule of interest is released from the complex into the cytoplasm free of lipids (Zelphati, O. and Szoka, Jr., F. C. (1996) Proc. Natl. Acad. Sci. USA 93, 11493-11498) and escaping lysosomal degradation. The noncovalent nature of these complexes is a major advantage of the liposome strategy as the delivered protein is not modified and therefore is less likely to lose its activity. Other cationic lipid systems used for introduction of molecules into cells include PULSin™ (Polyplus Transfection, distributed by Genesee Scientific, 8430 Juniper Creek Lane, San Diego, Calif. 92126) and SAINT-PhD (Synvolux Therapeutics B. V., L. J. Zielstraweg 1, 9713 GX Groningen, The Netherlands). PULSin™ contains a proprietary cationic amphiphile molecule that forms non-covalent complexes with proteins and antibodies. Complexes are believed to be internalized via anionic cell-adhesion receptors and are released into the cytoplasm where they disassemble. The process is non-toxic and delivers functional proteins. SAINT-PhD consists of a proprietary cationic pyridinium amphiphile and a helper lipid. Upon mixture of SAINT-PhD with the protein a particle of approximately 200 nm in diameter is formed. In this particle the protein is enwrapped by at least one bilayer of lipids. Furthermore, in the complex formed only non-covalent interactions are present between SAINT-PhD and the protein. The cationic amphiphiles on the surface of the particle have high affinity for the negatively charged cell surface. Upon fusion or entrapment of the particle the protein is released into the cytoplasm of the cell. The proteins delivered by SAINT-PhD are functional and unmodified.

Molecules can also be introduced into cells or nuclei through cell or nuclear membrane permeabilization, for example, by use of digitonin or Streptolysin O. Streptolysin O can form pores up to the size of 35 nm in the plasma membrane of mammalian cells, which is generally lethal to the cell (Bhakdi et al., Adv Exp Med Biol. 1985; 184:3-21; Bhakdi et al., Infect Immun. 1985 January; 47(1):52-60; Walev et al., Proc Natl Acad Sci USA. 2001 Mar. 13; 98(6):3185-90; Walev et al., FASEB J. 2002 February; 16(2):237-9). However, transient low-dosage treatment with Streptolysin O in the absence of calcium ions allows the transient formation of membrane pores that are large enough to allow the passive diffusion of proteins. These pores are subsequently repaired upon addition of calcium ions, resulting in viable cells. Streptolysin O has been used to introduce molecules including anti-sense oligonucleotides and functional proteins into a cell (Fawcett et al., Exp Physiol. 1998 May; 83(3):293-303; Walev et al., supra). In one embodiment, Streptolysin O can be used to permeabilize the cellular membrane to allow the cells to be loaded with cellular extracts of another cell type. For permeabilization with Streptolysin O, cells are typically incubated in Streptolysin O solution (see, for example, Maghazachi et al., FASEB J. 1997 August; 11(10):765-74) for 15-30 minutes at room temperature. For digitonin permeabilization, cells are suspended in culture medium containing digitonin at a concentration of approximately 0.001-0.1% and incubated on ice for 10 minutes. After permeabilization, the cells are typically washed by centrifugation at 400×g for 10 minutes. Typically, this washing step is repeated twice by resuspension and sedimentation in PBS. Cells are typically kept in PBS at room temperature until use. Alternatively, the cells can be permeabilized while placed on coverslips to minimize the handling of the cells and to eliminate the centrifugation of the cells, which in some instances can improve the viability of the cells. The permeabilized cells are then contacted with the desired substances (e.g., cell extract, purified protein, etc.). After the procedure, the cellular membrane of cells treated with Streptolysin O can be resealed in the presence of calcium.

Molecules can also be introduced into cells or cell nuclei by linkage to a protein transduction domain (PTD) or nuclear translocation domain or nuclear localization signal such as those already mentioned. For example, a protein may be expressed as a fusion protein that includes a PTD or NLS. Additionally, a molecule to be introduced into a cell may be covalently or noncovalently linked to a PTD or NLS using other means known in the art, e.g., using a chemical linker, avidin-biotin linkage, streptavidin-biotin linkage, Protein A/Fc linkage, Protein G/Fc linkage, etc. Exemplary PTDs that may be used for introduction of molecules of interest into cells are described, under the heading “Fusion Proteins,” infra. Multiple PTDs (which may be the same or different) may be linked to a molecule to be introduced into a cell.

Another means of introducing molecules into a recipient cell or nucleus comprises the introduction of or contacting with cytoplasm blebs derived from a donor cell.

The recipient cell can be of any species and may be heterologous to the donor cell, e.g., amphibian, mammalian, avian, with mammalian cells being preferred. Especially preferred recipient cells include human and other primate cells, e.g., chimpanzee, cynomolgus monkey, baboon, other Old World monkey cells, caprine, equine, porcine, ovine, and other ungulates, murine, canine, feline, and other mammalian species.

Exemplary methods of introducing donor cell cytoplasm into a recipient cell include microinjection, contacting donor cells with liposomal encapsulated cytoplasm, and enucleating the donor cell and incubating the recipient cell with a donor cell cytoplasmic extract. For example, this can be effected by microsurgically removing part or all of the cytoplasm of a donor cell with a micropipette and microinjecting such cytoplasm into that of a recipient cell. It may also be desirable to remove cytoplasm from the recipient cell prior to such introduction. Such removal may be accomplished by well known microsurgical methods. Alternatively, the cytoplasm and/or telomerase or telomerase DNA can be introduced using a liposomal delivery system.

In one embodiment, a polypeptide can be provided in the recipient cell media by being produced and secreted by engineered cells. For example, feeder cells may be engineered to express and secrete one or more desired reprogramming polypeptides. Optionally, engineered cells are physically separated from the recipient cells, e.g., by a selective barrier which may contain pores that allow diffusion of the reprogramming polypeptides but are too small for cells to pass through. Secretion of the reprogramming polypeptides may be effected through means known in the art, such as by fusion to a secretion signal. For example, a protein may be fused to or engineered to comprise a signal peptide, or a hydrophobic sequence that facilitates export and secretion of the protein. Whatever method is used to provide reprogramming proteins and other reprogramming agents in the cell media, those reprogramming agents can then be introduced into the recipient cells by any of the foregoing methods, preferably by linkage to a protein transduction domain, cell permeabilization, and/or addition of cationic lipids.

In exemplary embodiments, a recipient cell or nucleus may be contacted with the reprogramming composition more than once and/or for a prolonged incubation period, for example for at least one day, at least one week, at least one month, at least three months, at least one year, or longer. A suitable duration and/or number of times to contact with the reprogramming composition may be determined through routine experimentation, for example by measuring one or more reprogramming-associated phenotypes described herein (e.g., expression of one or more genes associated with the target cell type, detecting the methylation state and/or histone acetylation state of the promoters of one or more genes associated with the target cell type, measuring the expression of an engineered reporter construct indicative of reprogramming to adopt the target cell type, and detecting morphology characteristic of the target cell type).

Treatment of Disease

Many diseases resulting from the dysfunction of cells may be amenable to treatment by the administration of reprogrammed cells. These include diseases of cardiac, neurological, endocrinological, vascular, retinal, dermatological, and muscular-skeletal systems, and other diseases.

Transforming a patient's own cells into a desired cell type that needs replacement, reprogramming will permit the generation of autologous, genetically matched cells that would not be subject to immune rejection on transplantation. Additionally, reprogrammed cell lines created according to the methods described herein can be a source of cells for transplantation.

In one embodiment, a reprogrammed cell is prepared from a patient's relative, such as a histocompatible relative. For example, for treatment of a patient having a genetic disorder, a reprogrammed cell may be prepared from a transplant-compatible relative who does not have the genetic disorder.

Preferably the cells are histocompatible with the individual recipient, such that the undesirable use of immunosuppression is decreased or eliminated. For example, histocompatible cells may be obtained from the patient, from a donor related to the patient, or an unrelated donor. Optionally the cells are genetically modified so alter their histocompatibility profile, such that they are more compatible with the patient.

A “bank” of different reprogrammed cell lines can be created by the methods herein, and can provide sources of cells for therapeutic transplant that are highly histocompatible with human or non-human patients in need of cell transplants. For example, a reprogrammed cell line may be established from a patient, a relative of the patient, or an unrelated individual. In a more specific embodiment a bank of different reprogrammed cells may be produced for potential use in cell therapies or transplantation therapy as the need may arise. Thus, an object of the present disclosure to prepare a collection of reprogrammed cell lines that can be used for therapeutic transplant. Certain of the cell lines are homozygous for at least one histocompatibility antigen, which is particularly desirable to increase the number of individuals histocompatible with a given line. In addition these cells may be genetically modified before, during or after reprogramming so as to eliminate a genetic defect that is correlated to a specific disease so as to preclude disease relapse in transplantation therapies using cells produced using the subject reprogramming methods such as pancreatic cells or bone marrow cells.

Among reprogrammed cells that can be produced by these methods are such sought-after cells as cardiomyocytes, neurons, oligodendrocytes, retinal pigment epithelium, insulin-producing cells, skeletal myoblasts, smooth muscle cells, and others. Such cells and tissues would satisfy an unmet medical need for tissue and organ repair and could be generated to decrease the risk of immune rejection either through banking a variety of genetically diverse cell lines or via patient-specific reprogramming.

The cells may be used in various methods known in the art, including being injected into a patient, grown on a scaffold and surgically implanted, directly applied to the site of an injury, etc.

For example, reprogrammed cells of the present disclosure may be used as a source of muscle cells for prevention or treatment of diseases involving loss or dysfunction of muscle cells or that can be prevented or treated by providing a source of muscle cells. Exemplary disease and conditions that may be prevented or treated include myopathy, chronic fatigue syndrome, fibromyalgia, ALS (Lou Gehrig's disease), MS (multiple sclerosis), Central Fibrillar Shy-Magee Syndrome, Thomsen disease, Statin-associated myopathy, muscular dystrophy (including Duchenne, Becker, limb girdle, congenital, facioscapulohumeral, myotonic, oculopharyngeal, distal, and Emery-Dreifuss), spinal muscular atrophy, Brown-Vialetto-Van Laere syndrome, Fazio-Londe (FL) syndrome, inclusion body myositis, polymyalgia rheumatica, dermatomyositis, polymyositis, rhabdomyolysis, compartment syndrome, muscle atrophy, nemaline myopathy, multi/minicore myopathy, centronuclear myopathy (or myotubular myopathy), mitochondrial myopathies, familial periodic paralysis, inflammatory myopathies, metabolic myopathies, glycogen storage diseases, lipid storage disorder, myositis ossificans, myoglobinurias, alcoholic myopathy, and trauma. Additional exemplary diseases and conditions that may be prevented or treated are cardiomyopathies including intrinsic and extrinsic cardiomyopathies, such as genetic, acquired, mixed, metabolic/storage, inflammatory, endocrine, toxicity, neuromuscular, nutritional, and ischemic cardiomyopathies, which include without limitation: hypertrophic cardiomyopathy, arrhythmogenic right ventricular cardiomyopathy, isolated ventricular non-compaction, mitochondrial myopathy, dilated cardiomyopathy, restrictive cardiomyopathy, Takotsubo cardiomyopathy, Loeffler endocarditis, amyloidosis, cardiomyopathy associated with hemochromatosis, Chagas disease, diabetic cardiomyopathy, cardiomyopathy associated with hyperthyroidism, cardiomyopathy associated with chemotherapy, alcoholic cardiomyopathy, muscular dystrophy, and ischemic cardiomyopathy. Additional exemplary diseases and conditions that may be prevented or treated include diseases involving smooth muscle, such as vascular diseases (including atherosclerosis, arteriosclerosis, arteriolosclerosis, and atherosclerosis), stenosis, aneurysm, claudication, infarction, cirrhosis, fibrosis of the lung, asthma, allergies, or generalized smooth-muscle disease. The reprogrammed cells of the present disclosure may also be used to ameliorate the loss of muscle associated with a disease or condition such as cachexia, anorexia, Dejerine Sottas syndrome, inactivity (e.g., bed rest or immobilization of a limb or joint as part of a treatment for a musculoskeletal injury), congestive heart failure, chronic obstructive pulmonary disease, liver disease, starvation, aging, or resulting from microgravity or weightlessness. In this context, the term “diseases” generally encompasses diseases and other conditions. Similarly, the terms “treat,” “treatment,” and generally includes prevention, treatment, palliative care (including treatment of symptoms caused by other treatments), amelioration of symptoms, prophylactic treatments, and the like.

As a further example, neurodegenerative disease frequently include neuronal cell loss, and, because of the absence of endogenous repopulation, effective recovery of function is either extremely limited or absent. Reprogrammed cells of the present disclosure may be used as a source for cell-based therapies for neurodegenerative disease diseases, including Parkinson's disease, Amyotrophic Lateral Sclerosis, Multiple System Atrophy, Tay-Sachs Disease, Alzheimer's disease, Alexander's disease, Alper's disease, Ataxia telangiectasia, Batten disease, Bovine spongiform encephalopathy (BSE), Canavan disease, Cerebral palsy, Cockayne syndrome, Corticobasal degeneration, Creutzfeldt-Jakob disease, Familial Fatal Insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spinocerebellar ataxia, Spinal muscular atrophy, Steele-Richardson-Olszewski disease, Tabes dorsalis, and Toxic encephalopathy.

Also, the subject methods may be used for the production of autologous grafts, e.g., skin grafts, which can be used in the case of tissue injury or elective surgery.

Yet another application of the present application is for treating the effects of chronologic and UV-induced aging on the skin. As skin ages, various physical changes may be manifested including discoloration, loss of elasticity, loss of radiance, and the appearance of fine lines and wrinkles. It is anticipated that such effects of aging may be alleviated or even reversed by topical application of reprogramming factor-containing compositions. For example, reprogramming agent-containing compositions, optionally further including telomerase or a telomerase DNA construct, can be packaged in liposomes to facilitate internalization into skin cells upon topical application. Also, it may be advantageous to include in such compositions compounds that facilitate absorption into the skin, e.g., DMSO. These compositions may be topically applied to areas of the skin wherein the effects of aging are most pronounced, e.g., the skin around the eyes, the neck and the hands.

The present disclosure also provides methods for alleviating the effects of aging. Just as mammalian cells have a finite life-span in tissue culture, they similarly have a finite life-span in vivo. This finite life-span is hypothesized to explain at least some of the undesired effects of aging (including decreased immune-system function). The present disclosure provides methods to alleviate the effects of aging by providing a source of cells for cell and tissue therapy. For example, reprogrammed cells can then be introduced into the individual. This can be used, e.g., to rejuvenate the immune system of an individual. Such rejuvenation should be useful in the treatment of diseases thought to be of immune origin, e.g., some cancers, age-associated decrease of immune function, etc.

Genetically Modified Cells

Another significant application of the present disclosure is for gene therapy. To date, many different genes of significant therapeutic importance have been identified and cloned. Moreover, methods for stably introducing such DNAs into desired cells, e.g., mammalian cells and, more preferably, human somatic cell types, are well known. Also, methods for effecting site-specific insertion of desired DNAs via homologous recombination are well known in the art.

Another exemplary genetic modification is introduction of a conditional “suicide gene” such as a suicide gene under a conditional promoter. For example, if for any reason the transplanted cells react in a in a way that can harm the recipient, expression of the suicide genes can be induced to kill some or all of the transplanted cells. Use of inducible suicide genes in this manner is known in the art. Suitable suicide genes include genes encoding HSV thymidine kinase and cytodine deaminase, with which cell death is induced by gancyclovir and 5-fluorocytosine, respectively. A suicide gene may also be placed under the control of a lineage-specific promoter, such that cells in which that promoter is activated are eliminated.

Exemplary genetic modifications include modifications that change a cell's histocompatibility profile, for example, by alteration of one or more HLA genes, such as by allele replacement or deletion. For example, such methods may be used to generate a “bank” of cell lines suitable for transplant into patients having different histocompatibility profiles.

Other exemplary genetic modifications decrease immune rejection responses, such as modifications that cause expression proteins that inhibit immune rejection responses such as CD40-L (CD154 or gp139), modifications that prevent generation of an antigen that can trigger an immune rejection response, e.g. a glycosylated antigen expressed by porcine or other animal cells.

Exemplary genetic modifications include replacement of a disease-associated or disease-susceptible genomic sequence with a wild-type or disease-resistant sequence. For example, introduction of a gene or replacement of alleles of a gene contained in the cell line that provides resistance to disease (e.g., an HIV-resistant allele of CCR5, such as the CCR5 delta 32 allele; a cancer-resistant allele of an oncogene or tumor suppressor gene). Another exemplary genetic modification is introduction of increased copies of the tumor suppressor gene p53, which has been shown to decrease cancer incidence and improve health-span in mice (Garcia-Cao et al., EMBO J. 2002 Nov. 15; 21(22):6225-35). Other exemplary genetic modifications include those that eliminate mutations correlated to neoplastic, autoimmune, or other genetic diseases such as cystic fibrosis, sickle cell anemia, breast cancer, prostate cancer and the like. Another exemplary genetic modification is introduction of increased copy number of the DSCR1 gene and/or the Dyrk1a, which are genes located on human chromosome 21 that have been implicated in the greatly decreased cancer incidence in individuals affected with Down's Syndrome (Baek et al., Down's syndrome suppression of tumor growth and the role of the calcineurin inhibitor DSCR1. Nature advance online publication 20 May 2009|doi:10.1038/nature08062). Certain embodiments include an increased copy number of a tumor suppressor gene (such as p53 or Rb). Other embodiments include increased copy number and/or modifications that result in increased expression of certain genes that are expected to promote health and/or fight disease including genes involved in DNA repair, antioxidant defense gene (e.g., a superoxide dismutase such as SOD1, SOD2, SOD3, a catalase), genes involved in DNA repair or chromosome maintenance, telomerase genes, etc.

Other embodiments include introduction of exogenous genes expected to provide health benefits to a cell transplant recipient. For example, certain embodiments can include introduction of genes encoding enzymes capable of selectively degrading pathogenic material that accumulates with age and has been implicated in age-associated diseases. These pathogenic materials include cholesterol, oxidized cholesterol, and 7-ketocholesterol (implicated in heart disease and stroke), beta-amyloid plaques and neurofibrillary tangles in the brain (implicated in Alzheimer's disease), lipofuscin such as A2E in the retinal pigment epithelium (implicated in age-related macular degeneration), and extracellular matrix protein cross-links due to exposure of the tissue to high sugar levels such as carboxymethyllysine, carboxyethyllysine, Argpyrimidine, and other advanced glycation end products (implicated in diabetes).

Cells of the present disclosure can also be genetically modified to provide a therapeutic gene product that the patient requires, e.g., due to an inborn error of metabolism. Many genetic diseases are known to result from an inability of a patient's cells to produce a specific gene product. For example, a cell may be genetically modified to synthesize enhanced amounts of a gene product required by a patient. For example, hematopoietic stem cells that are genetically altered to produce and secrete adenosine deaminase can be prepared for transplant to a patient suffering from adenosine deaminase deficiency.

Preferably, the aforementioned genetic modifications are targeted modifications that avoid the risk of insertion at a site in the genomic DNA that disrupts normal cellular function, such as disruption of growth control that can cause neoplastic transformation. Alternatively, non-targeted methods may be used, such as using a recombinant retrovirus, and the insertion site(s) can then be identified to evaluate suitability of that cell for a particular use, for example by disqualifying cells where the insertion has the potential to disrupt a cell's normal growth control, and/or contains undesired viral sequences.

The present methods will also be beneficial in situations wherein the expression of a desired gene product or phenotype is dependent upon the expression of different DNA sequences, or for gene research involving the interrelated effects of different genes on one another. Moreover, it is anticipated that the present methods will become very important as the interrelated effects of the expression of different genes on others becomes more understood.

Methods of Identifying and Verifying Dedifferentiated Cells.

Candidate reprogrammed cells can be identified and verified using various methods. These methods include examining cell and colony morphology; determining whether the cells exhibit functional characteristics of the target cell type; determining whether cells express characteristic markers of the target cell type; and comparing gene methylation to the target cell type.

Additionally, candidate reprogrammed cells can be analyzed to determine whether unwanted genetic and/or epigenetic alterations are present. For example, cells may be karyotyped, such as by cytological methods (including classic and spectral karyotyping methods) and/or by sequencing-based methods (e.g. digital karyotyping). Cells can also be tested to determine whether loss of heterozygosity has occurred, for example by comparing the genome-wide SNP profile between untreated cells and reprogrammed cells, with loss of heterozygosity indicating that potentially undesired recombination events have occurred (though in some instances loss of heterozygosity may be desired, for example to eliminate a particular unwanted allele). Additionally, cells can be tested to determine whether particular undesired sequences are present, e.g., undesired viral sequences, nucleic acids encoding reprogramming factors to which a cell has been exposed, mycoplasma and other pathogens, etc. Cells can also be tested to detect aberrant expression of oncogenes and/or tumor suppressors. Cells can also be tested for unwanted genome sequence modification by partial or full genome sequencing, which is optionally targeted to the sequences of particular genes (e.g. genes involved in growth regulation). Cells can also be tested for undesired epigenetic changes, such as undesired histone modification (Jenuwein et al., Science. 2001 Aug. 10; 293(5532):1074-80; Strahl et al., Nature. 2000 Jan. 6; 403(6765):41-5; Turner, Nat Cell Biol. 2007 January; 9(1):2-6).

Fusion Proteins

In certain exemplary embodiments of the present methods and compositions include fusion proteins. These fusion proteins contain domains or regions of proteins which are arranged differently than they are found in nature, for example by joining portions of different polypeptides.

Exemplary protein translocation domains (PTDs) include the HIV transactivating protein (TAT) (Tat 47-57) (Schwarze and Dowdy 2000 Trends Pharmacol. Sci. 21: 45-48; Krosl et al. 2003 Nature Medicine (9): 1428-1432). For the HIV TAT protein, an amino acid sequence sufficient to confer membrane translocation activity corresponds to residues 47-57 (YGRKKRRQRRR, SEQ ID NO: 1) (Ho et al., 2001, Cancer Research 61: 473-477; Vives et al., 1997, J. Biol. Chem. 272: 16010-16017). This sequence alone can confer protein transduction activity when attached to another polypeptide. The TAT PTD may also be the nine amino acids peptide sequence RKKRRQRRR (SEQ ID NO: 2) (Park et al. Mol Cells 2002 (30):202-8). The TAT PTD sequences may be any of the peptide sequences disclosed in Ho et al., 2001, Cancer Research 61: 473-477 (the disclosure of which is hereby incorporated by reference herein), including YARKARRQARR (SEQ ID NO: 3), YARAAARQARA (SEQ ID NO: 4), YARAARRAARR (SEQ ID NO: 5) and RARAARRAARA (SEQ ID NO: 6). Other proteins that contain PTDs include the herpes simplex virus 1 (HSV-1) DNA-binding protein VP22 and the Drosophila Antennapedia (Antp) homeotic transcription factor (Schwarze et al. 2000 Trends Cell Biol. (10): 290-295). For Antp, amino acids 43-58 (RQIKIWFQNRRMKWKK, SEQ ID NO: 7) represent are sufficient for protein transduction, and for HSV VP22 the PTD is represented by the residues DAATATRGRSAASRPTERPRAPARSASRPRRPVE (SEQ ID NO: 8). Alternatively, HeptaARG (RRRRRRR, SEQ ID NO: 9), or even larger poly-arginine peptides (e.g., having eight, nine, ten, eleven, etc. up to twenty or more arginine residues) or artificial peptides that confer transduction activity may be used as a PTD of the present disclosure.

In additional embodiments, the PTD may be a PTD peptide that is duplicated or multimerized. In certain embodiments, the PTD is one or more of the TAT PTD peptide YARAAARQARA (SEQ ID NO: 4). In certain embodiments, the PTD is a multimer consisting of three of the TAT PTD peptide YARAAARQARAYARAAARQARAYARAAARQARA (SEQ ID NO: 10). A protein that is fused or linked to a multimeric PTD, such as, for example, a triplicated synthetic protein transduction domain (tPTD), may exhibit reduced lability and increased stability in cells. Such a construct may also be stable in serum-free medium and in the presence of hES cells.

### EXAMPLES

### Example 1

This example describes cell extracts used as a source of reprogramming agents, which may be used to reprogram somatic cells. While not intending to be limited by theory, it is hypothesized that a given cell type contains regulatory factors (including transcription factors) that determine its gene expression profile and identity; thus, exposing one type of cell to regulatory factors derived from a different type of cell can redirect the gene expression pattern and identity toward a different type of cell. Additionally, this conversion can be promoted by specific inducing factors, cell culture conditions, Chromatin remodeling agents, and/or Transcription Modifiers. Unless stated otherwise, cell extract generation and recipient cell permeabilization are performed essentially as described in Agarwal, “Cellular Reprogramming” (2006) Methods Enzymol. 420: 265-283 which is incorporated by reference herein in its entirety.

Reprogramming extracts were generated using clonal, myoblastic C2C12 cells. C2C12 cells are a subclone of a myoblast line established from a normal adult C3H mouse leg muscle. They have been used extensively as a model to study in vitro myogenesis and cell differentiation due to their characteristic rapid differentiation in low serum to form extensive contractile myotubes expressing characteristic muscle proteins. They provide a source of defined, committed (to the muscle lineage) but not fully differentiated, differentiating and differentiated cell extracts for use in reprogramming. Efficiency of reprogramming using extracts collected from cells at different stages of the differentiating process can be readily determined using these methods.

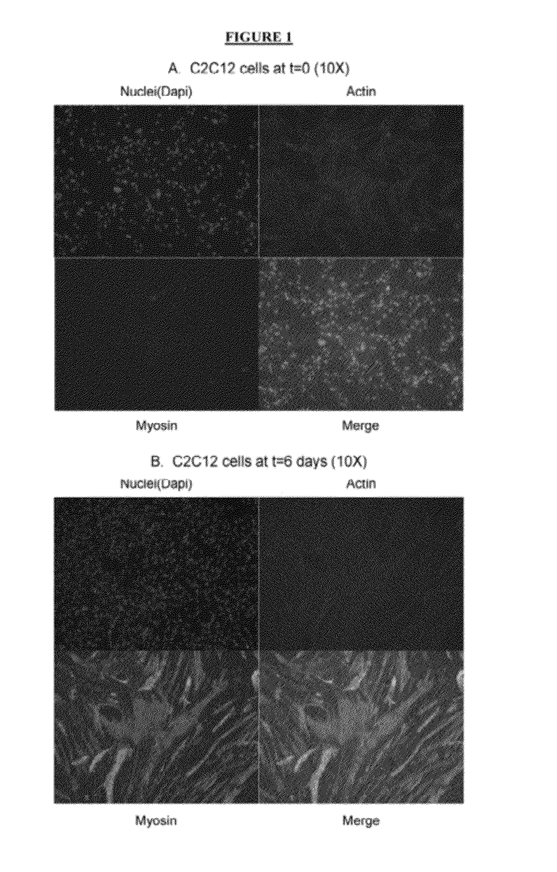

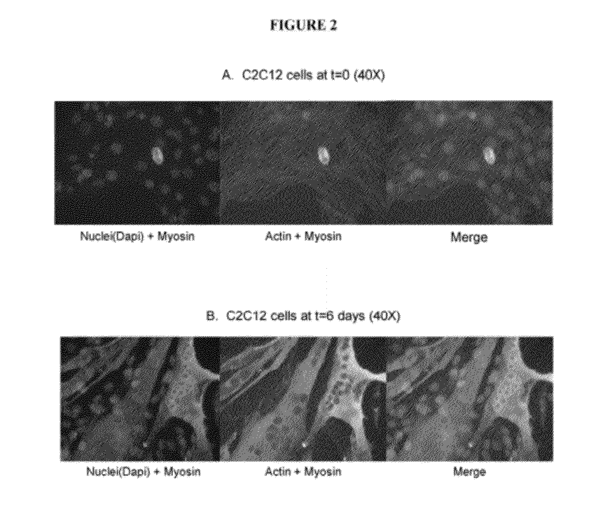

FIGS. 1 and 2 illustrates the differentiation of mouse myoblastic C2C12 cells to myotubes. Maintaining the C2C12 myoblastic cells under conditions that maintain healthy cultures with low differentiation background and high differentiation potential may improve the efficiency of reprogramming reactions. As shown in FIG. 1, before the differentiation process was initiated (at time zero), the C2C12 cells were grown in media containing 20% serum and the cultures contain mono-nucleated cells with an extremely low number staining positive on immuno fluorescence for the late skeletal muscle marker protein, sarcomeric Myosin heavy Chain (MHC). After induction of differentiation (by switching to media containing 2% serum; time=6 days shown), the majority of the cells show an elongated, multi-nucleate, fused sarcomeric phenotype of differentiated myotubes, which stain strongly positive with the anti-myosin antibody. This can be seen even more clearly at higher magnification in FIG. 2. This effect was usually apparent even at three days post differentiation. Reprogramming extracts were collected at this time point also.

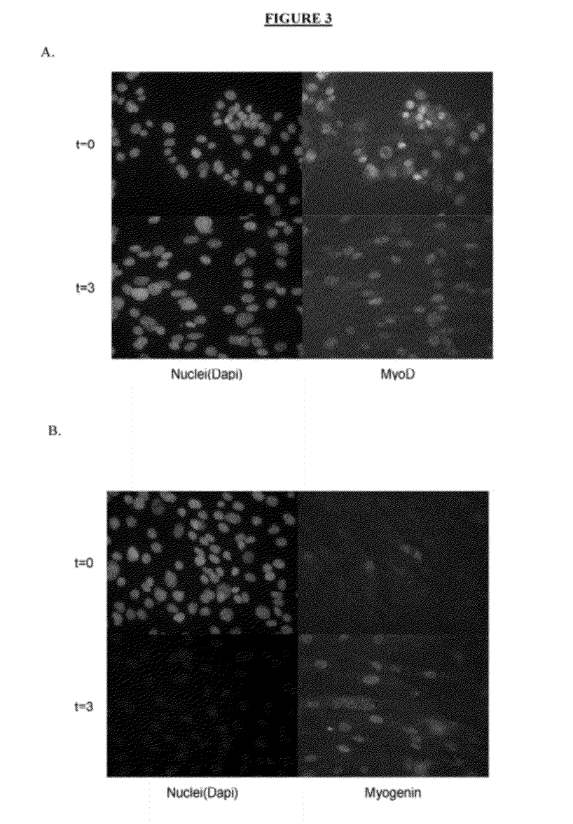

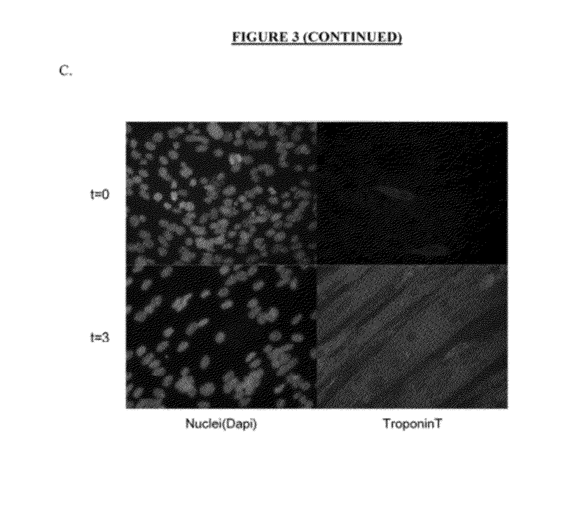

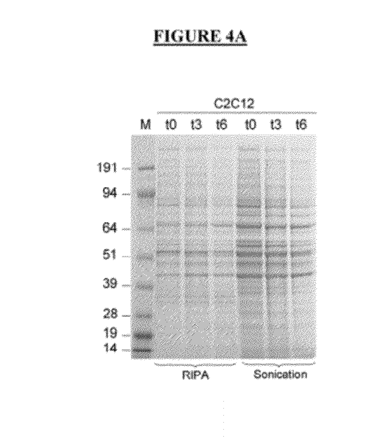

As additional indicators to characterize these cell extracts and the reprogramming effects observed, we examined this muscle differentiation system for additional muscle specific markers, both early and late. FIGS. 3A, 3B and 3C depict the characterization of the C2C12 muscle differentiation system using antibodies against the muscle transcription factors MyoD and Myogenin; and, Troponin T, the skeletal muscle specific subunit of Troponin, which facilitates the cell movement generating interactions between actin and myosin. Depicted are cells at time zero (t=0) and 3 days after differentiation, when they have generated visible myotubes (t=3). MyoD and Myogenin belong to a family of helix-loop-helix (HLH) transcription factors that are though to play an important role in the regulation of muscle cell development. MyoD is an early marker of muscle cells in a committed state. As illustrated in FIG. 3A, MyoD was expressed in the nuclei of myoblastic C2C12 cells at time zero and through differentiation to myotubes at t=3. MyoD is thought to be a ‘master regulator’ of transcription that is involved in triggering a cascade of muscle specific transcription (for instance, of Myogenin and Troponin T) during myogenesis. Myogenin is an induced gene having little or no expression during early commitment (FIG. 3B: t=0) but expression was detected in the nucleus when the cells are induced to differentiate (t=3). Troponin T, a cytoplasmic protein, was absent in early myoblastic cells (FIG. 3C: t=0) but induced highly on differentiation (t=3). Having identified conditions for the induction of differentiation and characterized the system, we collected lysates of C2C12 cells at different stages of differentiation at the time-points indicated in the experiments below (typically at time zero, time=3 days and time=6 days, post induction of differentiation). Obtaining extracts from cell-cycle synchronized populations is also envisioned, as reprogramming may be more effective using extracts from synchronized cells.

### Example 2

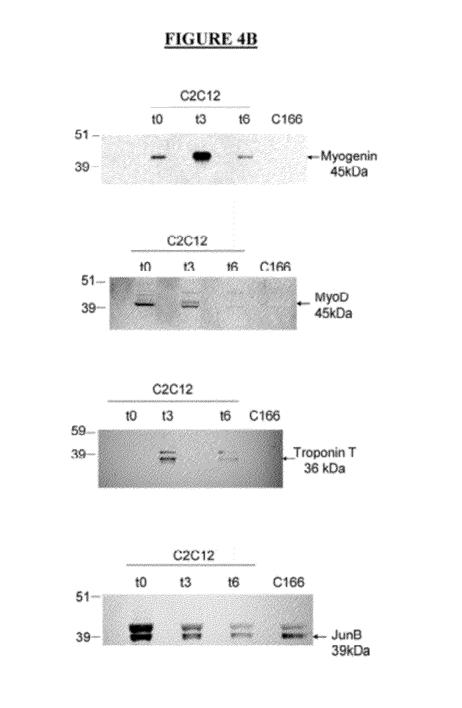

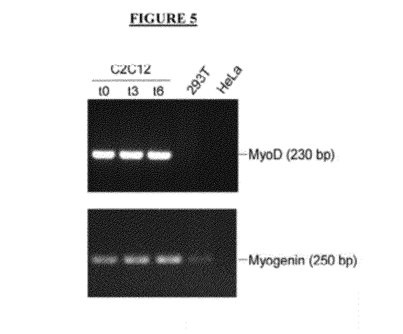

This example describes biochemical characterization of reprogramming cell extracts. Inspection and characterization of the input cell extracts can reveal the state of the reprogramming agents contained therein and show the extent of protein degradation and efficiency of release and so lublization of cell proteins. FIG. 4A depicts a Coomassie Blue stained SDS-PAGE gel loaded with whole cell extracts of different time points of differentiating C2C12 cells, prepared either by a traditional highly efficient detergent based lysis procedure using RIPA buffer or by the methods describe in Example 1 (which avoid the use of detergents). The protein profiles of the released proteins were indistinguishable using both methods, demonstrating that the sonication method yields high levels of intact, soluble protein.

Similarly, FIG. 4B depicts a series of immunoblots that examine the presence of marker proteins in the various differentiating priming C2C12 cell extracts. For each gel, equal amounts of total cell lysates of the C2C12 cells or of C166 endothelial cells (which served as a non-muscle negative control cell line) were resolved by SDS-PAGE and immunoblotted for muscle specific marker proteins Myogenin, MyoD and Troponin T or the general transcription factor JunB. These proteins were expressed and intact in the extracts at the expected time points. In agreement with our observations by immuno fluorescence (FIG. 3, above), the expression of the muscle differentiation specific transcription factor Myogenin was relatively low at time=0 but more highly induced at t=3. As expected, the transcription factor MyoD, however, was expressed at time=0 equally well as at t=3. Both factors were apparently downregulated at t=6. The induced cytoplasmic protein Troponin T, was clearly absent at t=0 and then induced at t=3 and t=6. JunB was, as expected, expressed at all time points in the C2C12 cells (although it may be downregulated with differentiation) and also in the control C166 cells. These results ascertain the intact nature of our whole cell extracts and ensure that they include characteristic proteins (such as the transcription factor MyoD), which might be expected to be important for reprogramming.

### Example 3

This example describes gene expression analysis assays (RT-PCR) to detect cell reprogramming.

A hallmark of a particular cell type is its gene expression profile. Reverse-Transcription of the expressed RNA in a cell, combined with Polymerase Chain Reaction aided amplification of the signal (RT-PCR), was used to detect the expression levels of these genes and provide a method to detect cell reprogramming. This example illustrates RT-PCR assays for detection of muscle specific genes in C2C12 cells (FIG. 5). Total RNA prepared from C2C12 cells at different stages of differentiation (t=0, t=3, t=6) or from 293T cells or HeLa cells were used in an RT-PCR reaction with primers specific to the muscle specific genes, MyoD or Myogenin. As shown, both the primer pairs yielded major RT-PCR products of the expected sizes, predominantly expressed in the myoblastic C2C12 cells. There was some detection of background level amplification from 293T cells, however, in the case of Myogenin. Myogenin expression was also detectable in C2C12 cells at T=0 (this may have been due to low levels of differentiation already initiated in the cell cultures). RT-PCR conditions for the detection of late muscle specific genes Troponin T and Myosin Heavy Chain, and for the hES cell marker Oct-4, were also established. Due to its relatively high sensitivity, gene expression analysis using RT-PCR was used to detect expression of marker genes in permeablized and potentially reprogrammed recipient cells.

### Example 4

This example demonstrates reprogramming of permeabilized recipient cells using extracts from C2C12 cells. The recipient cells used in these experiments were 293T cells and primary human dermal fibroblasts (NHDFs). Recipient cells were treated with reprogramming extracts under varying conditions (including variation in media/serum cell growth conditions before/after the priming reaction; concentration of cellular extracts used; timing and sequence of addition). Reprogramming efficiency may be improved by optimizing these conditions through routine experimentation for a given recipient cell type and source of reprogramming agents.

Methods

Cell extracts were introduced into a recipient cell by reversible permeabilization of recipient cells using the pore-forming, calcium sensitive bacterial toxin Streptolysin O (SLO) and exposure of these cells to reprogramming cell extracts. Limited and transient exposure of cells to low doses of SLO in the absence of calcium ions allows the formation of membrane pores large enough to allow the passive diffusion of proteins up to the size of 100 kilodaltons, but not large enough for organelles. Subsequently, reversal of this membrane permeabilization by adding calcium ions allows the membrane to repair itself and the resealed cells are viable and can proliferate. During the permeabilization process, the recipient cell was incubated with whole cell extracts or nuclear or cytoplasmic extracts of a desired cell type (“donor cell”), and (in some experiments) in the presence of permeabilization/reprogramming promoting agents such as an energy generating system and cytoskeletal disruptors. The whole cell extract is believed to provide regulatory factors that are taken up by the permeabilizing recipient cell. The plasma membrane was then resealed, the cells were allowed to recover and cultured further, in conditions conducive to desired reprogramming. Consequently, gene expression profiles of the recipient cells become altered to more closely resemble the donor cells. The resultant changes in gene expression profiles may arise over time, and may be further promoted by subsequent rounds of treatment with donor cell extract.

Fluorescently conjugated proteins (70 kDA Rhodamine-dextran or 68 KDa Rhodamine-albumin) were used in some experiments to conveniently monitor the efficiency of cell permeabilization and uptake of exogenous factors. We found that the efficiency of SLO mediated membrane permeabilization and uptake of proteins can be sensitive to several factors including the density of the cells, use of adherent versus suspension cell cultures, the concentration of SLO, SLO activation, length of exposure to SLO and exogenous factors, the quality of the cell extracts (if cell extracts are used), and time given for resealing of membrane pores and recovery. These factors can be routinely optimized for a given cell type.

Results

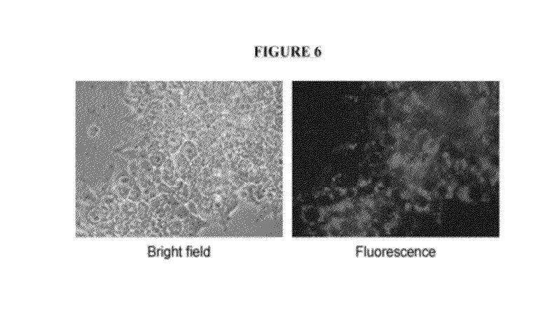

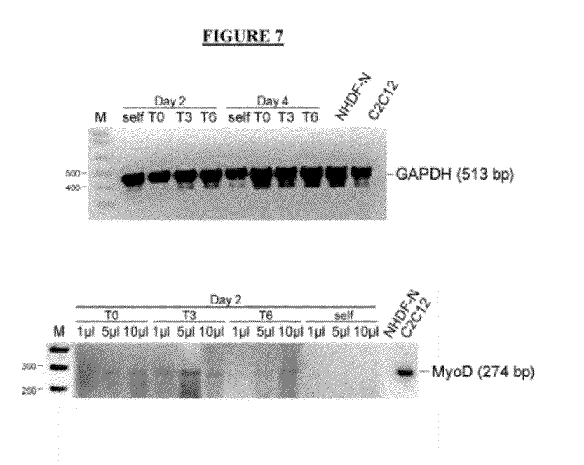

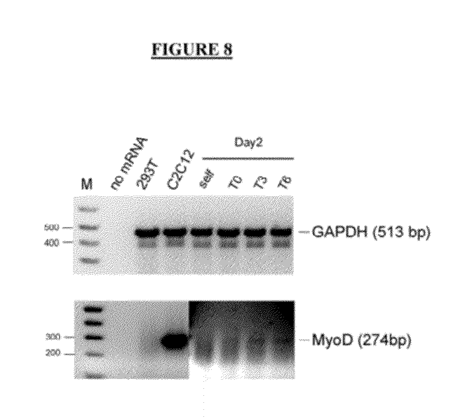

FIG. 6 depicts one example of cell reprogramming. Permeabilized 293T cells were primed with differentiating C2C12 extracts (extract t=3 depicted) in the presence of the fluorescent marker protein, Rhodamine-Albumin, in order to monitor the permeabilization and survival of the cells. As shown, the cells appeared to have survived well and took up robust amounts of the fluorescent protein (pictures at Day 4 after permeabilization). These cells proceeded to proliferate and could be passaged to collect later time points for analysis. Similar results were obtained with primary human dermal fibroblasts (NHDFs). Total RNA was collected from the recipient cells at different times following the reprogramming reaction, and RT-PCR was performed to test for muscle specific gene expression (as well as for expression of a ubiquitously expressed housekeeping gene as an internal control). In the representative experiment shown in FIG. 7, primary neonatal Normal Human Dermal Fibroblast (NHDF-N) cells were incubated with either NHDF cell extract (“self” control) or extracts of C2C12 cells collected at different stages of differentiation: at time zero (T0), time=3 days (T3) or time=6 days (T6), post induction of differentiation, and total RNA was collected at different times post permeabilization and analyzed by RT-PCR. The lower panel shows detection the 274 bp MyoD product in RT-PCR reactions performed on increasing amounts of isolated total RNA. MyoD expression was detectable in permeabilized NHDF treated with C2C12, indicating at least partial reprogramming and adoption of a muscle cell phenotype. The level of this transcript in the C2C12 extract incubated NHDF cells appeared to be low (relative to control C2C12 cells) but was detected reproducibly in independent reactions. Control NHDF cells treated with NHDF cell extract (“self” controls) did not show expression of the MyoD PCR product. Positive control C2C12 cells showed robust expression of MyoD as expected. The upper panel depicts RT-PCR amplification with primers specific to the ubiquitously expressed house keeping gene GAPDH (samples from Day 2 and 4 shown) as a positive control. As expected, all samples yielded a specific 513 bp product, confirming the integrity of the isolated RNA and of the RT-PCR reactions.

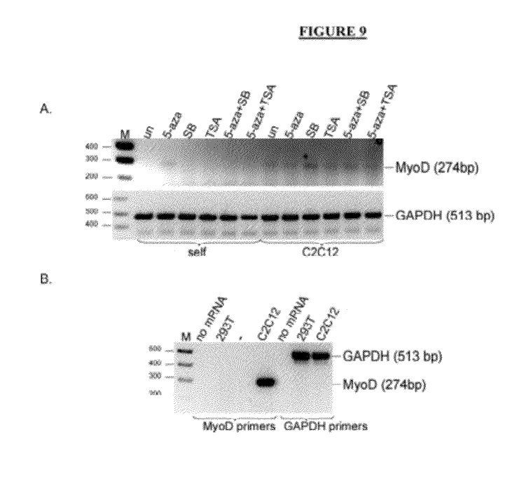

Similar results were obtained with human kidney 293T cells (FIG. 8). Cells were treated with reprogramming extracts (or control 293T “self” extracts) as above. After permeabilized 293T cells were treated with the C2C12 extract, the specific 274 bp RT-PCR product indicative of MyoD expression was reproducibly detectable (FIG. 8, lower panel), indicating at least partial reprogramming and adoption of a muscle cell phenotype. The level of MyoD expression in these cells was low (relative to control C2C12 cells) but reproducible. Control reactions with the GAPDH specific primers yielded the expected intact 513 bp specific product, except the control reaction without RNA (FIG. 8, upper panel).

Thus, permeabilization and incubation of non-muscle cells (NHDF or 293T cells) with myogenic C2C12 extracts leads to detectable early expression of a muscle specific marker.

### Example 5

### Enhancement of Reprogramming Using Transcription Modifying/Chromatin Remodeling Agents on Recipient Cells

This example describes use of transcription modifying and chromatin remodeling agents to enhance the efficiency of reprogramming. The DNA methyltransferase inhibitor 5 azacytidine and the Histone DeAcetylase (HDAC) inhibitors Sodium Butyrate and Trichostatin A were tested. DNA methylation (the addition of a methyl group to the 5 position of cytosine) is an epigenetic process that affects chromatin structure, transcriptional suppression and transposition. 5 azacytidine, a nucleoside analog, can activate gene expression. It is believed to act by inhibiting DNA methyltransferases. HDAC inhibitors can modulate gene expression by causing histone hyperacetylation and resultant chromatin relaxation. These agents may also cause cell cycle arrest and/or apoptosis. The tolerable dosages of these agents can readily be determined for a given cell type.

We first titrated the dosage and time of treatment of cells to determine toxicity. In the tolerable range identified, we observed a reduction in the growth rate of the cells and while thus treated cells could tolerate incubation with cell extracts, treatment with these agents, particularly Sodium Butyrate, did cause some apparent toxicity, post permeabilization.

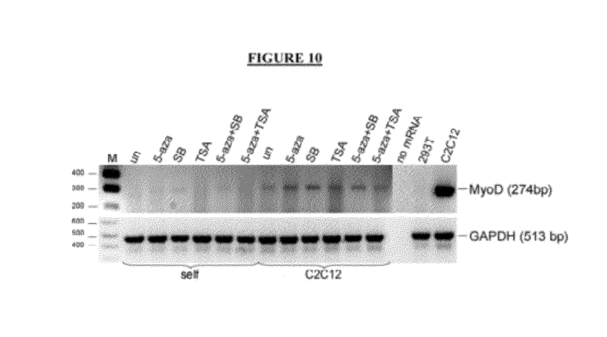

We next turned to testing the effects of these treatments on reprogramming potential by evaluating reprogramming of 293T or primary NHDF-N cells incubated with cell extracts from the C2C12 muscle differentiating system by gene expression analysis (RT-PCR). In the case of examining the effects of transcription modifying agents, several variations of treatment plans were envisioned. The agents can be applied before or after permeabilizing the cells (or both) and for different durations of time, as long as the cells survive the treatment. Plausibly, different treatment designs could potentially lead to different effects. Starting with the tolerable time and dosage conditions identified above, we tested several treatment regimens. In the experiment illustrated in FIG. 9, 293T cells were permeabilized and incubated with either 293T cell extract (“self” control) or extracts of C2C12 cells. The primed cells were then either left untreated (un) or treated with 5 azacytidine (5-aza), Sodium Butyrate (SB), Trichostatin A (TSA) or combinations thereof. Total RNA was collected at different times post priming and analyzed by RT-PCR with primers specific for GAPDH or the muscle specific gene, MyoD, as above. Again, as seen in the upper panel of FIG. 9A, incubation of even untreated 293T cells (un) with extracts from C2C12 cells (but not with 293T “self” extract) resulted in the detection of the MyoD specific 274 bp RT-PCR product. Treatment of these C2C12 extract exposed cells with the transcription modifying agents post priming, did not, however, appear to alter the level detection of this RNA, except perhaps in the case of Sodium Butyrate which may have caused a small increase in expression. Moreover, in this experiment, 5-azacytidine appeared to lead to a low level of expression of MyoD in cells primed with control “self” extract (relative to cells treated with C2C12 extract), which would be consistent with its role as a global non-specific transcriptional enhancer.

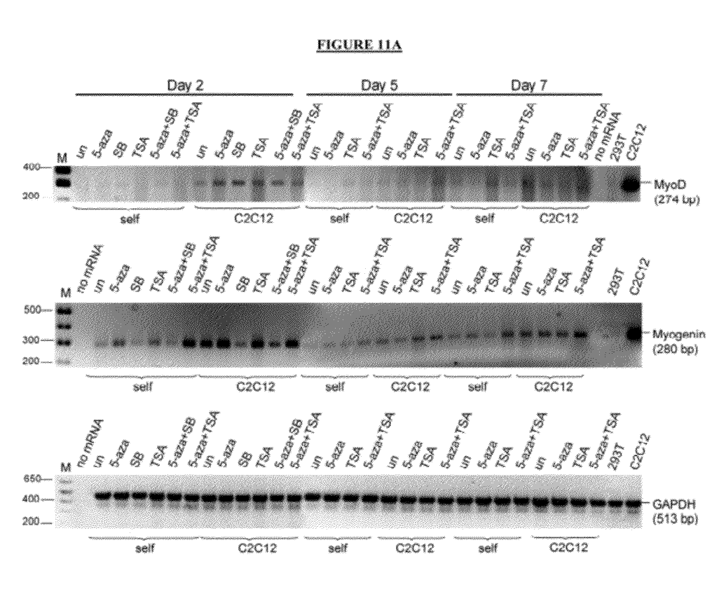

In another treatment regimen (FIG. 10), 293T cells were left untreated (un) or treated with 5 azacytidine (5-aza), Sodium Butyrate (SB), Trichostatin A (TSA) or combinations thereof, for 48 hours. The cells were then permeabilized and incubated with either 293T cell extract (“self” control) or extracts of C2C12 cells (C2C12). Total RNA was collected at different times post permeabilization and analyzed by RT-PCR with primers specific for GAPDH or the muscle specific gene, MyoD. As in the preceding experiments, incubation of even untreated 293T cells (un) with extracts from C2C12 cells (but not with “self” extract) resulted in the detection of MyoD specific RT-PCR product. Treatment of these C2C12 extract incubated cells with 5-aza, SB, TSA or combinations thereof before permeabilization, appeared to enhance the level detection of this RNA (relative to cells treated with C2C12 extract alone) to a low but reproducible extent. In this experiment, 5-aza and SB appeared to lead to a low level of expression of MyoD in cells incubated with control “self” extract also (relative to cells treated with C2C12 extract), which would again be consistent with their role as global non-specific transcriptional enhancers. Treatments 5-aza, SB, TSA or combinations thereof subsequent to the permeabilization reaction did not appear to enhance the level of MyoD expression.

As described previously, MyoD is known to be an early marker of muscle cells in a committed state. In addition to MyoD, muscle cells express several other genes, at various stages of their differentiation, which mark them to be muscle. To test whether later-stage markers of muscle differentiation were induced by the reprogramming extract, we tested for expression of Myogenin, Myosin Heavy Chain and Troponin T using RT-PCR (along with testing for expression of GAPDH and MyoD in these same samples). Also included were expression analyses of recipient cells at later time points post permeabilization and incubation with extracts.

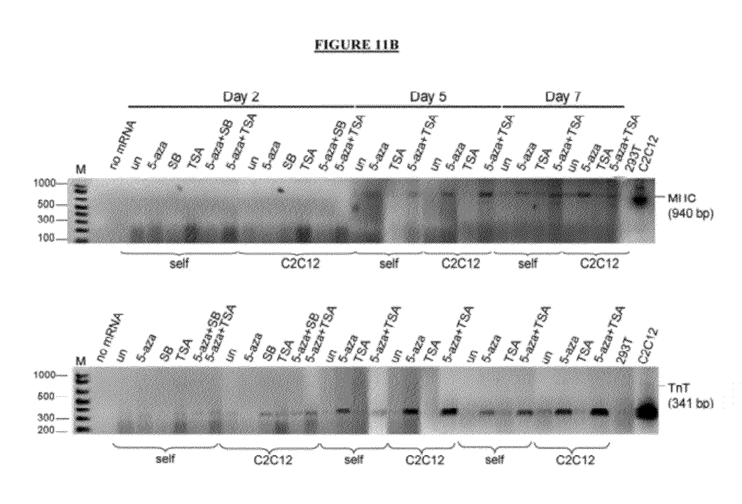

As seen in the lower panel of FIG. 11A (samples from Day 2, Day 5 and Day 7 post permeabilization shown), all reactions with the GAPDH specific primers yield the expected intact 513 bp specific product (except for the control reaction without RNA). In this experiment, Sodium Butyrate (SB) treated cells did not survive beyond Day 2 post permeabilization and are hence not included in samples from later time points. As described above, incubation of even untreated 293T cells (un) with extracts from C2C12 cells resulted in the clear detection of MyoD specific RT-PCR product in Day 2 samples and treatment of these C2C12 extract primed cells with 5-aza, SB, TSA or combinations thereof appeared to enhance the level detection of this RNA slightly, in the Day 2 samples (upper panel). Detection of MyoD RNA was noticeably reduced in samples from the later timepoints, in both treated ‘self’-incubated cells and untreated/treated ‘C2C12-incubated’ cells. Although still discernable on Day 7, especially in untreated or 5-azacytidine and TSA treated, C2C12-extract-incubated cells, the level of MyoD RNA appeared to decrease over time.

The middle panel of FIG. 11A depicts RT-PCR analysis of the same RNA samples with primers specific to Myogenin. Analysis of RNA from C2C12 cells (positive control) yielded the detection of an expected very robust Myogenin specific 280 bp RT-PCR product which was detected as a very faint background signal in RNA from unpermeabilized 293T cells also. In Day 2 samples, permeabilization and incubation of untreated 293T cells (un) with even 293T “self” extract resulted in the detection of the non-specific induction of a Myogenin specific 280 bp RT-PCR product, which was significantly increased on incubating the cells with extracts from C2C12 cells. Further, treatment of the cells (both self-incubated and, to a larger extent, C2C12-incubated) with transcription modifying agents led to still further increases in the expression of Myogenin, with the largest effect seen on treatment with 5-azacytidine and TSA. The Myogenin transcript, although reduced in samples from later timepoints, persisted very clearly and to a larger extent than MyoD RNA. Again, the level in C2C12 incubated cells and further in 5-azacytidine plus TSA treated cells appeared to be the highest, suggesting prolonged expression of muscle-specific transcripts, and hence reprogramming, of the cells.

Expression of Myosin Heavy Chain (MHC) and Troponin T were measured in the same cells and gave similar results (FIG. 11B). RT-PCR analysis of RNA from control C2C12 cells yielded the detection of an expected robust MHC specific 940 bp product which was not detected in RNA from unpermeabilized 293T cells (upper panel). Unlike the early Muscle markers MyoD and Myogenin, RNA for this late marker protein was not detected in Day 2 samples at all. Instead, the transcript was first seen in self-extract treated and to a larger extent, C2C12-extract incubated cells on Day 5. The signal persisted clearly in Day 7 samples. For Troponin T (FIG. 11B, lower panel), control C2C12 cells contain an expected specific 341 bp RNA species which was not detected in unpermeabilized 293T cells. In Day 2 samples, permeabilization and incubation of untreated 293T cells (un) with “self” extract, and more so with C2C12 extracts, resulted in the detection of a Troponin T specific RT-PCR product, which was increased on treatment with transcription modifiers, suggesting both non-specific and specific induction of the transcript. However, in samples from later timepoints, the Troponin T transcript appeared to become more abundant and more specific to modifier treated (especially 5-azacytidine and 5-azacytidine plus TSA treated) and C2C12 extract incubated cells. Taken together, the initial presence and low level persistence of MyoD transcripts (relative to C2C12 cells) and the stronger and timely progression of expression of downstream muscle markers in these permeabilized and transcription modifier treated cells suggests at least transient induction of a muscle transcription program in these cells.

### Example 6

### Use of Specific Muscle Cell Inducing Factors to Promote Reprogramming

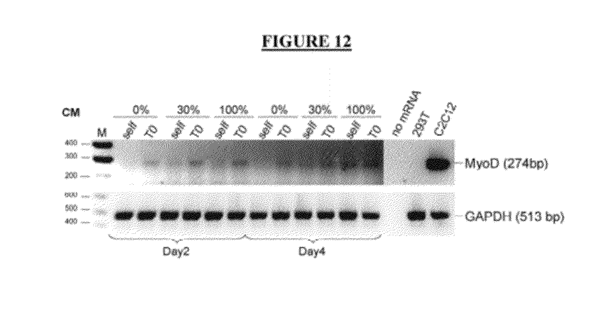

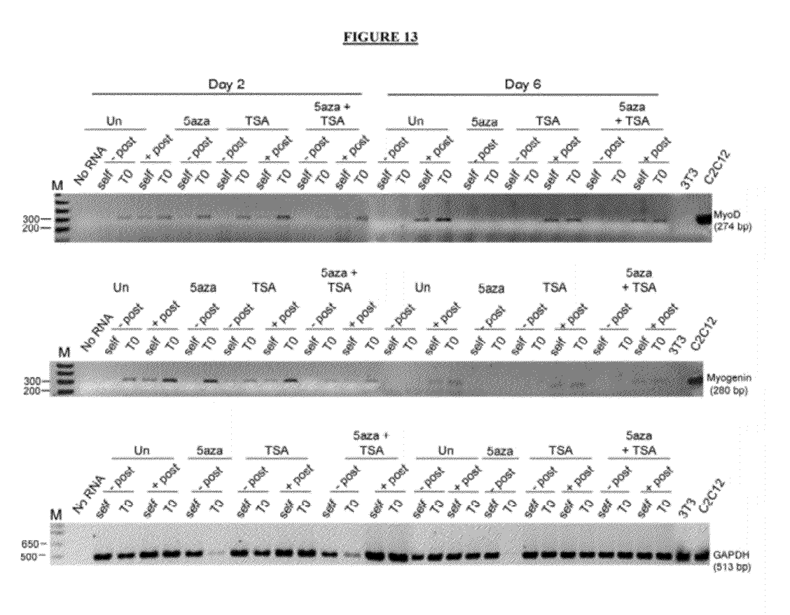

This example describes tests of additional conditions to further enhance reprogramming of non-muscle cells into muscle cells. To test whether autocrine factors secreted by C2C12 cells may further promote reprogramming to a muscle fate, we collected conditioned media from healthy, exponentially growing C2C12 cells. 293T cells were permeabilized and incubated with either 293T cell extract (“self” control) or extracts of C2C12 cells (T0). Immediately post the reprogramming incubation, the cells were allowed to recover in either 100% regular media (C2C12 media with reduced (5%) serum), 70% regular media supplemented with 30% C2C12 conditioned media or 100% C2C12 conditioned media. Total RNA was then collected at different times post permeabilization and analyzed by RT-PCR with primers specific for GAPDH or the muscle specific gene, MyoD. As above, even in the absence of conditioned media, 293T cells treated with extracts from C2C12 cells (but not with 293T “self” extract) expressed low levels of MyoD (relative to C2C12 cells), indicated by presence of the MyoD specific 274 bp RT-PCR product (FIG. 12). The MyoD specific product continued to be detected in RNA samples harvested 4 days post priming. Treatment of the recipient cells with C2C12 conditioned media, both 30% and 100%, led to the detection of expression of MyoD even in cells incubated with control (self) extracts, and, this signal was enhanced on incubation with C2C12 extracts. Thus, C2C12 cells conditioned media appears to contain factors that can induce 293T cells to express muscle specific transcripts. The combination of conditioned media and/or other inducing factors, priming the cells with C2C12 extracts and treatment with transcription modifying agents could potentially further enhance reprogramming.

### Example 7

This example reports results of extended examination of several variations and combinations of treatment plans with inducing agents and transcription modifying agents.

A number of growth and differentiation factors have been implicated in various stages of development/specification of muscle cells from early mesoderm, as well as in the differentiation of adult muscle stem cells, or satellite cells, to terminally differentiated muscle cells. Examples include Insulin like Growth Factor 1 (IGF-1), which is known to be involved in promoting the differentiation of muscle progenitor cells including muscle satellite cells, acidic Fibroblast Growth Factor (aFGF) and Activin A. In this example, we evaluated the use of these specific factors in promoting muscle reprogramming. Typically, after first testing the effect of these factors when added alone to our reprogramming reactions, we combined them incrementally with pre/post treatment of the permeabilized recipient cells with transcription modifiers and C2C12 conditioned media. These studies were then extended farther by investigating a range of combinations of these various treatments over extended periods of time. Though subject to some variability, the results indicate a clear pattern wherein treatment with inducing factors (conditioned media and/or specific factors) post permeabilization and incubation of the recipient cells with cell extracts, prolongs the presence of muscle specific transcripts in non-muscle cells. In addition, pre and/or post permeabilization, treatment of the cells with transcription modifiers augments this effect. As discussed above, some enhancing effects of inducing factors and transcription modifiers are also seen in permeabilized recipient cells that were incubated with control or ‘self’-extract.

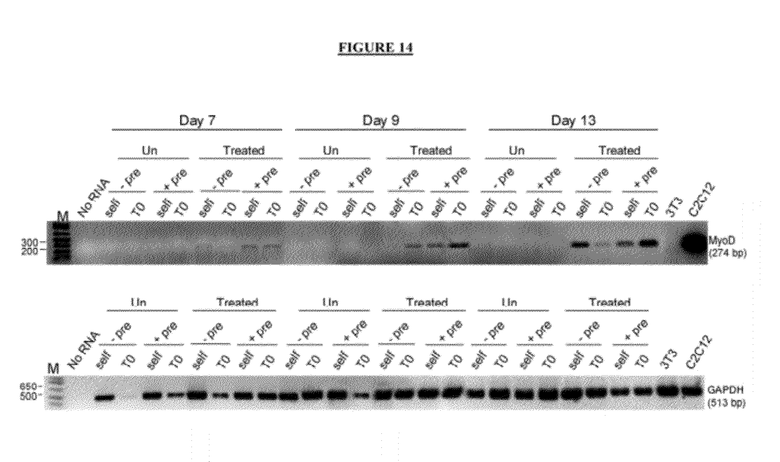

In this example, mouse NIH3T3 cells were treated with C2C12 muscle cell extracts, and results are shown for representative time points. NIH 3T3 cells were left untreated (un) or treated with 5 azacytidine (5-aza), Trichostatin A (TSA) or a combination of 5 azacytidine and TSA for 48 hours. The cells were then permeabilized and incubated with either NIH3T3 cell extract (“self” control) or extracts of C2C12 cells (T0). Immediately after the incubation, the cells were allowed to recover in the absence (−post) or presence (+post) of post-treatments (50% C2C12 conditioned media (CM) plus a continuation of respective pre-treatments). Total RNA was collected at different times after permeabilization and analyzed by RT-PCR with primers specific for GAPDH or the muscle specific genes, MyoD or Myogenin (FIG. 13). RT-PCR performed with control C2C12 cell RNA using MyoD (upper panel) or Myogenin (middle panel) specific primers resulted, respectively, in the detection of a 274 bp or 280 bp specific product that was not detected when analyzing RNA from 3T3 cells. However, permeabilization and incubation of even untreated 3T3 cells (un) with extracts from C2C12 cells (but not with 3T3 “self” extract), resulted in the clear detection of a MyoD-specific and a Myogenin-specific RT-PCR product in Day 2 samples, indicating the presence of MyoD and Myogenin RNA in these recipient cells. Interestingly, effects seen of pre and/or post treatment of the cells with transcription modifiers and conditioned media were very similar for both MyoD and Myogenin, including, and most notably, clear correlation of the detection of both RNA species in Day 6 samples with post-treatment and in both, self-incubated and C2C12-incubated cells. Thus, NIH3T3 cells exhibit the presence of muscle specific MyoD and Myogenin RNA when permeabilized and incubated with C2C12 cell extracts, and they can be induced to express MyoD and Myogenin by transcription modifier treatments and/or exposure to C2C12 conditioned media. Additionally, prolonged expression of MyoD and Myogenin appears to be greatly enhanced by post treatment with, at least, C2C12 conditioned media.

FIG. 14 depicts one example of testing the effect of including Insulin like Growth Factor 1 (IGF-1), acidic Fibroblast Growth Factor (aFGF) and Activin A in the treatment of 3T3 cells, post incubation with C2C12 cell extracts. In the experiment shown, 3T3 cells were left untreated (−pre) or pretreated (+pre) with 5 azacytidine (5-aza) and TSA for 2 days. The cells were then permeabilized and incubated with either 3T3 cell extract (“self” control) or extracts of C2C12 cells (T0). Immediately post the priming incubation, the cells were allowed to recover in the absence (Un) or presence (Treated) of post treatments (50% conditioned media with 5-aza and TSA (CM+A+T) with added IGF-1, aFGF and Activin A). One set of cells was split when it became confluent (Day 7), to generate additional sets to analyze at later time points (Day 9 and Day 13). Total RNA was collected from the cells at different times post permeabilization and analyzed by RT-PCR with primers specific for GAPDH or the muscle specific gene, MyoD. As shown in the lower panel of FIG. 14 (samples from Day 7, 9 and 13 post incubation shown), all reactions with the GAPDH specific primers yielded the expected 513 bp specific product (except for the control reaction without RNA). The presence of the MyoD specific 274 bp signal, correlated, at these later time points of assay, with treatment of the cells, post priming, which is consistent with the pattern observed above and additional experiments (not shown). Thus, in cells that were not pretreated, but were post treated (−pre and treated) or were both pre and post treated (+pre and treated), the MyoD transcript persisted to the 13 days post priming assayed, in most cases in both self-incubated and C2C12 extract-incubated cells. Persistent MyoD expression may be due to the continuous stimulation by transcription modifiers/conditioned media and specific muscle-inducing factors. MyoD expression persisted even through passaging and further growth of the cells. Collectively, thus, our observations suggest that the most consistent and sustained effect was seen on combining pretreatment with transcription modifiers and post treatment with inducing agents, specifically, muscle cell conditioned media and/or specific muscle inducing factors.

Each document cited herein is hereby incorporated by reference in its entirety to the extent that they are not inconsistent with the disclosures contained herein.

While the invention has been described by way of examples and preferred embodiments, it is understood that the words which have been used herein are words of description, rather than words of limitation. From the foregoing description, it will be apparent that variations and modifications may be made to the method described herein to adopt it to various usages and conditions. Changes may be made, within the purview of the appended claims, without departing from the scope and spirit of the invention in its broader aspects. Although the invention has been described herein with reference to particular means, materials, and embodiments, it is understood that the invention is not limited to the particulars disclosed. The invention extends to all equivalent structures, means, and uses which are within the scope of the appended claims.

1. A method of converting a somatic cell or somatic cell nucleus to a cell or nucleus of target type, wherein said target type is skeletal muscle, cardiac muscle, or smooth muscle, the method comprising:
(a) culturing the somatic cell or somatic cell nucleus in the presence of a reprogramming composition, thereby converting the somatic cell or somatic cell nucleus into a cell or nucleus of the target type, wherein said reprogramming composition comprises one or more reprogramming agents; and
(b) identifying a cell or cell nucleus of the target type that results from the culture of step (a).

2. The method of claim 1, wherein said target type is skeletal muscle.

3. The method of claim 1, wherein said reprogramming composition comprises an extract obtained from a muscle cell, an extract obtained from a myoblast cell, a muscle cell conditioned medium, or a myoblast cell conditioned medium.

4. The method of claim 3, wherein said extract or conditioned medium is obtained from cells of the same type as the target type.

5. The method of claim 3, wherein said extract is a whole cell extract, nuclear extract, or cytoplasmic extract.

6. The method of claim 3, wherein said reprogramming composition further comprises one or more exogenously added reprogramming agents or wherein the cell from which said extract is obtained has been modified to produce or express elevated levels of one or more reprogramming agents.

7. The method of claim 6, wherein said one or more exogenously added reprogramming agents are polypeptides selected from the group consisting of: MyoD, IGF-1, aFGF, and Activin A.

8. (canceled)

9. (canceled)

10. The method of claim 1, further comprising prior to, concurrently with, and/or subsequently to step (a), culturing said somatic or somatic cell nucleus cell in the presence of one or more DNA methyltransferase inhibitors.

11. (canceled)

12. The method of claim 1, further comprising prior to, concurrently with, and/or subsequently to step (a), culturing said somatic cell or somatic cell nucleus in the presence of one or more Histone DeAcetylase (HDAC) inhibitors.

13. (canceled)

14. The method of claim 1, wherein step (a) further comprises permeabilizing said somatic cell or somatic cell nucleus.

15. The method of claim 1, wherein step (a) comprises introducing said one or more reprogramming agents into said somatic cell or somatic cell nucleus by one or more methods selected from the group consisting of: microinjection, electroporation, fusion with a liposome, fusion with an enucleated donor cell, fusion or contact with a cytoplasmic bleb containing at least one reprogramming factor, and culturing said somatic cell or somatic cell nucleus in a medium containing said reprogramming composition and optionally containing a cell and/or nucleus entry agent.

16. The method of claim 15, wherein the cell and/or nucleus entry agent is selected from the group consisting of Streptolysin O, digitonin, a cationic amphiphile, and any combination thereof.

17. (canceled)

18. (canceled)

19. The method of claim 1, further comprising phenotypic monitoring of said somatic cell or somatic cell nucleus to measure its adoption of one or more phenotypes associated with the target cell or nucleus.

20. The method of claim 19, wherein said phenotypic monitoring comprises one or more measurements selected from the group consisting of detecting the expression of one or more genes associated with the target cell type, detecting the methylation state and/or histone acetylation state of the promoters of one or more genes associated with the target cell type, measuring the expression of an engineered reporter construct that is differentially active between said somatic cell and said target cell type, and detecting morphology indicative of said target cell type.

21. (canceled)

22. (canceled)

23. (canceled)

24. (canceled)

25. (canceled)

26. (canceled)

27. (canceled)

28. The method of claim 31, wherein said reprogramming polypeptide comprises at least one protein transduction domain that facilitates entry of the reprogramming polypeptide into the somatic cell or somatic cell nucleus.

29. (canceled)

30. (canceled)

31. The method of claim 1, wherein said reprogramming composition comprises one or more reprogramming polypeptides selected from the group consisting of: MyoD, IGF-1, aFGF, Activin A.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. A method of identifying a reprogramming agent that converts or enhances or potentiates the conversion of a somatic cell or somatic cell nucleus into a target type, wherein said target type is skeletal muscle, cardiac muscle, or smooth muscle, the method comprising:
(a) providing a candidate reprogramming composition comprising multiple candidate reprogramming agents;
(b) culturing a somatic cell or somatic cell nucleus that is not of the target cell type in the presence of said candidate reprogramming composition and culturing a control somatic cell or control somatic cell nucleus in the absence of said candidate reprogramming composition; and
(c) monitoring one or more phenotypes of said somatic cell or somatic cell nucleus to measure its adoption of one or more phenotypes associated with the target cell type, and identifying said candidate reprogramming composition as a reprogramming composition if said adoption of one or more phenotypes associated with the target cell type is significantly increased or accelerated relative to control cells or control cell nuclei cultured in the absence of said candidate reprogramming composition.

49. (canceled)

50. The method of claim 48, wherein said target cell type is skeletal muscle.

51. The method of claim 48, wherein said multiple candidate reprogramming agents comprise one or more genes selected from the group consisting of: MyoD, IGF-1, aFGF, and Activin A.

52. (canceled)

53. (canceled)

54. (canceled)

55. (canceled)

56. (canceled)

57. (canceled)

58. A cell therapy method which comprises administration to a subject in need thereof skeletal muscle, cardiac muscle, or smooth muscle cells produced according to the method of claim 1.

59. (canceled)

60. (canceled)

61. (canceled)

62. (canceled)
